(19) United States
(12) Patent Application Publication
(10) Pub. No.: US 2019/0328687 A1
(43) Pub. Date: Oct. 31, 2019

(54) Antibacterial Compounds

(71) Applicant: VICTORIA LINK LIMITED, Wellington (NZ)

(72) Inventors: Janine Naomi Copp, Nelson (NZ); David Francis Ackerley, Wellington (NZ)

(21) Appl. No.: 15/527,267

(22) PCT Filed: Nov. 17, 2015
(86) PCT No.: PCT/NZ2015/050192

§ 371 Date: May 16, 2017

(30) Foreign Application Priority Data
Nov. 17, 2014 (NZ) 702001

(51) Int. Cl. A61K 31/167; A61K 31/426; A61K 31/4015; A61P 31/04

(52) U.S. Cl. CPC A61K 31/167; A61P 31/04; A61K 31/4015; A61K 31/426

(57) ABSTRACT

The present invention relates to salicylamide compounds and compositions thereof effective in targeting growth of bacteria. The compounds and compositions of the present invention are particularly useful in, for example, the prevention or treatment of bacterial infection and the prevention, reduction or elimination of biofilm formation.

### TECHNICAL FIELD

The present invention relates to salicylamide compounds and compositions thereof effective in the prevention or treatment of bacterial infection caused by Gram positive bacteria. The present invention further relates to salicylamide compounds in combination with an efflux pump inhibitor, as well as compositions thereof, effective in the prevention or treatment of bacterial infection caused by Gram negative bacteria.

### BACKGROUND OF THE INVENTION

It is widely expected that the rise of multi-drug resistant bacteria will be the biggest health concern facing humans in the 21^{st} century (Boucher, H. W., Talbot, G. H., Bradley, J. S., Edwards, J. E., Gilbert, D., Rice, L. B., Scheld, M., Spellberg, B. and Bartlett, J. (2009). Bad Bugs, No Drugs: No ESKAPE! An Update from the Infectious Diseases Society of America. Clinical Infectious Diseases 48(1): 1-12; Piddock, L. J. (2012). The crisis of no new antibiotics—what is the way forward? Lancet Infect Dis. 12(3):249-53). Clinicians are already regularly faced with cases of antibiotic resistance, with previously simple to treat infections becoming more difficult and in some cases impossible to treat. Nearly all classes of antibiotics were discovered before 1970 and over the last 30 years no new major classes of antibiotics have been developed. Most advances recently have been within antibiotic classes, through the development of analogues to known antibiotics. However, resistance mechanisms have developed so that now whole classes of antibiotics are ineffective against certain bacteria.

To decrease the rate of antibiotic resistance, greater measures are being taken to limit the spread and incidence of infection in the first place, together with education on the proper use of antibiotics and limiting their use in ways that promote the development of infection. However, there is still a need for new antibiotics, in particular antibiotics that are effective against Gram negative pathogens, which represent a significant proportion of infectious disease burden.

Niclosamide (N-(2′-chloro-4′-nitrophenyl)-5-chlorosalicylamide) is a salicylanilide compound. Salicylanilides were identified as useful for killing snails following the screening of 20,000 compounds against the snail Biomphalaria glabrata in the 1950s and structural optimisation (Gönnert, R. (1961). Results of laboratory and field trials with the molluscicide Bayer 73.) Sun and Zhang (Sun, Z. and Zhang, Y. (1999). Antituberculosis activity of certain antifungal and antihelmintic drugs. Tubercle and Lung Disease 79(5): 319-320.) investigated antifungal and antihelmintic drugs for activity against Mycobacterium tuberculosis, broadly classified as a Gram positive bacteria, although it possesses “acid fast” cell wall characteristics of both Gram positive and negative bacteria. They found niclosamide to be very active against M. tuberculosis, with an MIC of 0.5-1 μg·mL⁻¹. Niclosamide was active against non-replicating M. tuberculosis grown in low oxygen conditions, which currently accounts for the lengthy treatment of M. tuberculosis infections. These authors did observe toxicity against macrophages grown in tissue culture. Salicylanilide analogues of niclosamide have been screened to further investigate their use in M. tuberculosis treatment (Kratky, M., Vinšová, J., Buchta, V., Horvati, K., Bösze, S. and Stolaříková, J. (2010). New amino acid esters of salicylanilides active against MDR-TB and other microbes. European journal of medicinal chemistry 45(12): 6106-6113; Krátký, M., Vinšovà, J., Novotná, E., Mandfková, J., Wsól, V., Trejtnar, F., Ulmann, V., Stolaříková, J., Fernandes, S. and Bhat, S. (2012). Salicylanilide derivatives block Mycobacterium tuberculosis through inhibition of isocitrate lyase and methionine aminopeptidase. Tuberculosis 92(5): 434-439.)

de Carvalho et al. also investigated niclosamide and the structural analogue nitazoxanide for efficacy against M. tuberculosis (de Carvalho, L. P. S., Darby, C. M., Rhee, K. Y. and Nathan, C. (2011). Nitazoxanide disrupts membrane potential and intrabacterial pH homeostasis of Mycobacterium tuberculosis. ACS medicinal chemistry letters 2(11): 849-854.). They showed that niclosamide and nitazoxanide uncoupled the membrane potential of M. tuberculosis, whereas a control, rifampicin, did not.

The potential of niclosamide as an indirect inhibitor of Gram negative pathogenesis was recently studied by Imperi et al., who screened FDA-approved drugs to identify any inhibitors of the quorum sensing system in Pseudomonas aeruginosa (Imperi, F., Massai, F., Ramachandran Pillai, C., Longo, F., Zennaro, E., Rampioni, G., Visca, P. and Leoni, L. (2013). New life for an old drug: the anthelmintic drug niclosamide inhibits Pseudomonas aeruginosa quorum sensing. Antimicrob Agents Chemother 57(2): 996-1005.). Of the drugs tested, niclosamide exhibited the highest anti-quorum sensing activity. Further analysis determined that niclosamide was able to inhibit the response to the quorum sensing signal rather than the synthesis of the signal molecule. However, the authors did not consider a directly toxic role for niclosamide, nor a possible role of drug efflux in defending cells against niclosamide and, according to the authors' data, niclosamide only appeared to be effective at inhibiting quorum sensing at micromolar concentrations or higher, suggesting that the drug was indeed being transported out of the cell.

Multi-drug efflux pumps can confer resistance to whole families of antibiotics. Efflux pumps are expressed in both Gram negative and Gram positive bacteria but are a more potent resistance mechanism in Gram negative bacteria. This is due to the double cell membrane which lowers drug permeability into the cytoplasm, and because drugs are immediately pumped out of the cell from the periplasm before reaching the cytoplasm. There are five known families of multidrug efflux transporters: small multidrug resistance protein family, multidrug and toxic compound extrusion protein family, major facilitator family, ATP-binding cassette family and the resistance nodulation cell division family (Paulsen, I. T., Chen, 3., Nelson, K. E. and Saier Jr, M. H. (2001). Comparative genomics of microbial drug efflux systems. Journal of molecular microbiology and biotechnology 3(2): 145-150.). The last three families are often located in the inner membrane of Gram negatives and work together with an outer membrane efflux protein, such as TolC, and a periplasmic efflux protein that enables the interaction between the inner and outer membrane transporters (Johnson, 3. M. and Church, G. M. (1999). Alignment and structure prediction of divergent protein families: periplasmic and outer membrane proteins of bacterial efflux pumps. Journal of Molecular Biology 287(3): 695-715.).

It is known that delivering an efflux pump inhibitor together with an antibiotic can increase the potency of an antibiotic even against strains that have been identified as resistant. Phenylalanine-arginine β-napthylamide (PAβN) is an efflux pump inhibitor which has a broad host and antibiotic range. Lomovskaya et al. (Lomovskaya, O., Warren, M. S., Lee, A., Galazzo, J., Fronko, R., Lee, M., Blais, 3., Cho, D., Chamberland, S., Renau, T., Leger, R., Hecker, S., Watkins, W., Hoshino, K., Ishida, H. and Lee, V. J. (2001). Identification and Characterization of Inhibitors of Multidrug Resistance Efflux Pumps in Pseudomonas aeruginosa: Novel Agents for Combination Therapy. Antimicrobial Agents and Chemotherapy 45(1): 105-116.) generated P. aeruginosa strains that over-expressed three efflux pumps known to confer resistance to fluoroquinolones in clinical isolates. They screened a library of synthetic and natural compounds by measuring growth inhibition in the presence of the antibiotic levofloxacin. PAβN was active against each of the efflux pumps and also active against AcrAB-TolC in E. coli. These authors demonstrated that the inclusion of the efflux pump inhibitor increased sensitivity to fluoroquinolones, reversed resistance to fluoroquinolones and decreased the frequency with which resistance developed.

Given the significant risk that antibiotic resistance presents to human and animal health, there is a need to develop novel drug/antibacterial approaches to treat and prevent infection. The present invention seeks to address this need by providing combination products comprising at least one salicylamide compound and at least one efflux pump inhibitor compound, or to at least provide a useful alternative to existing antibacterials.

Any reference to prior art documents in this specification is not to be considered an admission that such prior art is widely known or forms part of the common general knowledge in the field.

### SUMMARY OF THE INVENTION

In one aspect the present invention provides a salicylamide compound for use in treating or preventing a bacterial infection in a patient, wherein the bacteria causing infection comprises Gram positive bacteria.

In another aspect the present invention provides a salicylamide compound for use in preventing, reducing or eliminating formation of a bacterial biofilm, wherein the bacteria causing biofilm formation comprises Gram positive bacteria.

In a further aspect the present invention provides a pharmaceutical or biological composition comprising a salicylamide compound, together with an acceptable excipient, carrier or salt, for use in treating a bacterial infection in a patient or for use in preventing, reducing or eliminating formation of a bacterial biofilm, wherein the bacteria causing infection or biofilm formation comprises Gram positive bacteria.

In another aspect the present invention provides a method for treating or preventing a Gram positive bacterial infection comprising administering, to a patient requiring treatment, at least one salicylamide compound in an amount sufficient to treat or prevent the bacterial infection in the patient.

In yet a further aspect the present invention provides a method for reducing or eliminating formation of a bacterial biofilm comprising Gram positive bacteria, comprising administering at least one salicylamide compound in an amount sufficient to reduce or eliminate formation of the biofilm.

In another aspect the present invention provides use of a salicylamide compound in treating a bacterial infection in a patient or for preventing, reducing or eliminating formation of a bacterial biofilm, wherein the bacteria causing infection or biofilm formation comprises Gram positive bacteria.

In a further aspect the present invention provides the use of a salicylamide compound in the manufacture of a medicament for treating a bacterial infection in a patient or for use in preventing, reducing or eliminating formation of a bacterial biofilm, wherein the bacteria causing infection or biofilm formation comprises Gram positive bacteria.

In an example according to the above aspects of the present invention, the salicylamide compound is niclosamide or an analogue thereof, nitazoxanide or an analogue thereof, or any combination thereof.

In another example according to the above aspects of the present invention, the Gram positive bacteria is selected from one or more of the genus consisting of Staphylococcus, Listeria and Bacillus.

In another aspect the present invention provides a combination product comprising at least one salicylamide compound and at least one efflux pump inhibitor compound.

In yet another aspect the present invention provides a synergistic combination of at least one salicylamide compound and at least one efflux pump inhibitor compound.

In another aspect the present invention provides a composition comprising at least one salicylamide compound and at least one efflux pump inhibitor compound. In an example, the composition comprises synergistically effective amounts of the salicylamide compound and the efflux pump inhibitor compound.

In a further aspect the present invention provides a pharmaceutical or biological composition comprising at least one salicylamide compound and at least one efflux pump inhibitor compound, together with an acceptable excipient, carrier or salt.

The combination products or compositions according to the present invention may be used to treat or prevent a bacterial infection in a patient, or may be used to prevent, reduce or eliminate formation of a bacterial biofilm, where the infection or biofilm comprises Gram negative bacteria. The combination products or compositions according to the present invention may further comprise one or more bactericidal or bacteriostatic agents.

Accordingly, in another aspect the present invention provides the use of a combination of at least one salicylamide compound and at least one efflux pump inhibitor compound as a medicament or the use of a composition comprising at least one salicylamide compound and at least one efflux pump inhibitor compound as a medicament.

In another aspect the present invention provides a combination of at least one salicylamide compound and at least one efflux pump inhibitor compound for use in the preparation of a pharmaceutical composition.

In yet another aspect the present invention provides the use of a combination of at least one salicylamide compound and at least one efflux pump inhibitor compound for treating or preventing a bacterial infection in a patient.

In another aspect the present invention provides the use of a pharmaceutical composition comprising a pharmaceutically effective amount of at least one salicylamide compound and at least one efflux pump inhibitor compound for treating or preventing a bacterial infection in a patient, wherein the infection comprises Gram negative bacteria.

In a further aspect the present invention provides an anti-bacterial agent comprising at least one salicylamide compound and at least one efflux pump inhibitor compound. The anti-bacterial agent may be used to treat or prevent a bacterial infection in a patient, or it may be used to prevent, reduce or eliminate formation of a bacterial biofilm, in which Gram negative bacteria are present.

In an example, the biofilm causes infection in a wound and/or burn or causes an infection on or in an in-dwelling medical device, or the biofilm forms within preparative machinery for the food industry, on packaging used by the food industry, within storage tanks used for water or other liquids, or within machinery at water treatment plants.

In another aspect, the present invention provides use of at least one salicylamide compound and at least one efflux pump inhibitor compound in the manufacture of a medicament.

In yet another aspect the present invention provides a combination of at least one salicylamide compound and at least one efflux pump inhibitor compound for use in the manufacture of a medicament.

In yet another aspect the present invention provides a kit of parts comprising at least one salicylamide compound and at least one efflux pump inhibitor compound in separate unit dosage forms, together with instructions for use.

In another aspect the present invention provides a pharmaceutical composition comprising at least one salicylamide compound and at least one efflux pump inhibitor compound for treating or preventing a bacterial infection in a patient, wherein the infection comprises Gram negative bacteria.

In yet another aspect the present invention provides the use of at least one salicylamide compound and at least one efflux pump inhibitor compound in the manufacture of a medicament for treating or preventing a bacterial infection in a patient, wherein the infection comprises Gram negative bacteria.

In yet another aspect the present invention provides a method of treating or preventing a bacterial infection, comprising administering, to a patient requiring treatment, at least one salicylamide compound and at least one efflux pump inhibitor compound in amounts sufficient to treat or prevent the bacterial infection in the patient, wherein the infection comprises Gram negative bacteria.

In yet another aspect the present invention provides a method for protecting a bacterial cell against toxicity by at least one salicylamide compound, wherein the salicylamide compound includes one or more nitro groups, the method comprising increasing the expression and/or activity of at least one nitroreductase enzyme in the cell in an amount sufficient to protect against toxicity by the salicylamide compound.

In yet another aspect the present invention provides a method for treating or preventing a bacterial infection in a patient, wherein the bacteria have become resistant to treatment with a nitro-prodrug antibiotic, comprising administering to the patient at least one salicylamide compound, wherein the salicylamide compound includes one or more nitro groups, in an amount sufficient to treat or prevent infection. Optionally, the method further comprises administering at least one efflux pump inhibitor.

In yet another aspect the present invention provides a method for preventing, reducing or eliminating formation of a bacterial biofilm, wherein the bacteria have become resistant to treatment with a nitro-prodrug antibiotic, comprising administering at least one salicylamide compound, wherein the salicylamide compound includes one or more nitro groups, in an amount sufficient to prevent, reduce or eliminate formation of the biofilm. Optionally, the method further comprises administering at least one efflux pump inhibitor.

In yet another aspect the present invention provides a method for treating or preventing a bacterial infection in a patient, wherein the bacteria have become resistant to treatment with at least one salicylamide compound or the combination of at least one salicylamide compound and at least one efflux pump inhibitor compound, wherein the salicylamide compound includes one or more nitro groups, comprising administering to the patient a nitro-prodrug antibiotic in an amount sufficient to treat or prevent the infection.

In yet another aspect the present invention provides a method for preventing, reducing or eliminating formation of a bacterial biofilm, wherein the bacteria have become resistant to treatment with at least one salicylamide compound or the combination of at least one salicylamide compound and at least one efflux pump inhibitor compound, wherein the salicylamide compound includes one or more nitro groups, comprising administering a nitro-prodrug antibiotic in an amount sufficient to prevent, reduce or eliminate formation of the biofilm.

The present invention also contemplates co-administration of a nitro-prodrug and niclosamide so as to simultaneously target pro-drug resistant bacteria as well as prodrug sensitive bacteria.

Accordingly in yet another aspect the present invention provides a method for treating or preventing a bacterial infection in a patient, or for preventing, reducing or eliminating formation of a bacterial biofilm, comprising administering a nitro-prodrug antibiotic and niclosamide in an amount sufficient to treat or prevent the infection or to prevent, reduce or eliminate formation of the biofilm.

In an example, the nitro-prodrug antibiotic is selected from the group consisting of nitrofurantoin, nitrofurazone, metronidazole, tinidazole, furazolidone, misonidazole, etanidazole, nifurtimox, ornidazole, benznidazole, dimetridazole, ronidazole, RSU-1069, RB-6145, CB1954, EF3, EF5, HX4 and fluorinated misonidazole.

In yet another aspect the present invention provides a screening method to identify novel nitroreductase enzymes, the method comprising the steps of:

(i) performing a targeted or random mutagenesis of an existing nitroreductase gene thereby producing a nitroreductase variant gene library; and
(ii) transforming the variant gene library into Gram negative bacteria in which the tolC gene has been deleted or the tolC expression product has been inhibited and culturing the cells so that gene variant is expressed; and
(iii) administering at least one salicylamide compound to the transformed bacterial cells; and
(iv) screening for cells which lack susceptibility to salicylamide toxicity thereby identifying cells which express a novel form of the nitroreductase enzyme; and
(v) optionally purifying the nitroreductase enzyme.

In an example, the endogenous nitroreductase genes of the Gram negative bacteria have been knocked out or nitroreductase activity in the Gram negative bacteria has been reduced or eliminated.

In yet another aspect the present invention provides a screening method to identify novel nitroreductase enzymes from a preparation of environmentally sourced DNA, the method comprising the steps of:

(i) generating a bacterial gene library from that environmentally sourced DNA; and
(ii) transforming the gene library into Gram negative bacteria in which the tolC gene has been deleted or the tolC expression product has been inhibited and culturing the cells so that gene library is expressed; and
(iii) administering at least one salicylamide compound to the transformed bacterial cells; and
(iv) screening for cells which lack susceptibility to salicylamide thereby identifying cells which express a novel form of the nitroreductase enzyme; and
(v) optionally purifying the nitroreductase enzyme.

In an example, the endogenous nitroreductase genes of the Gram negative bacteria have been knocked out or nitroreductase activity in the Gram negative bacteria has been reduced or eliminated.

In another example, the environmentally sourced DNA is sourced from soil.

In yet another aspect the present invention provides a screening method to identify novel inhibitors of TolC, the method comprising the steps of:

(i) culturing Gram negative bacteria which express TolC in the presence of at least one salicylamide compound and a candidate inhibitor of TolC; and
(ii) screening for cells which are susceptible to salicylamide toxicity thereby identifying novel inhibitors of TolC.

In an example, the salicylamide compound and the efflux pump inhibitor compound provide a synergistic antibacterial effect.

In another aspect the present invention provides a composition comprising antibiotically effective amounts of:

(i) a salicylamide compound or a pharmaceutically acceptable salt thereof; and
(ii) an efflux pump inhibitor compound or a pharmaceutically acceptable salt thereof; wherein compounds (i) and (ii) are employed in proportions sufficient to produce a synergistic antibiotic effect.

In an example, the bacterial infection is a bacterial infection caused by one or more Gram negative bacteria.

In another example, the bacterial infection is caused by Pseudomonas aeruginosa, Klebsiella pneumoniae, Escherichia coli, Burkholderia multivorans, Pseudomonas syringae pv. actinidiae (Psa-V), Neisseria gonorrhoeae, Acinetobacter baumannii, Shigella species, Salmonella species or Enterobacter species.

In a further example, the salicylamide compound includes one or more nitro groups.

In an example the salicylamide compound is a salicylanilide compound, and may be substituted with one or more nitro groups.

In another example the salicylanilide compound is a compound of formula (I):

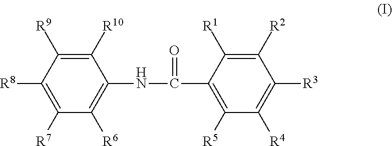

wherein:
R¹, R², R³, R⁴ and R⁵ are each independently selected from the group consisting of hydrogen, hydroxy and halogen, provided that at least one of R¹, R², R³, R⁴ and R⁵ is halogen and at least one of R¹, R², R³, R⁴ and R⁵ is hydroxy;
R⁶, R⁷, R⁸, R⁹ and R¹⁰ are each independently selected from the group consisting of hydrogen, nitro and halogen, provided that at least one of R⁶, R⁷, R⁸, R⁹ and R¹⁰ is halogen and at least one of R⁶, R⁷, R⁸, R⁹ and R¹⁰ is nitro;
or a pharmaceutically acceptable salt thereof.

In certain examples, any halogen in formula (I) is selected from the group consisting of chloro, fluoro and bromo. More preferably at least one halogen in formula (I) is chloro.

In an example, R⁸ in formula (I) is nitro. Alternatively, R⁹ is nitro. Alternatively, R¹⁰ is nitro. Alternatively, R⁷ is nitro.

In a related example, the salicylanilide compound is a compound selected from Table 1 below.

In a further related example, the salicylanilide compound is niclosamide having the structure:

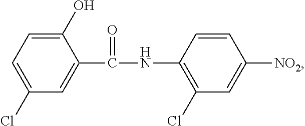 [SMILES: O=C(Nc1ccc([N+](=O)[O-])cc1Cl)c1cc(Cl)ccc1O]

or a pharmaceutically acceptable salt thereof.

In an example, the pharmaceutically acceptable salt is an ethanolamine salt or a piperizine salt.

Alternatively the salicylamide compound is selected from the group consisting of:

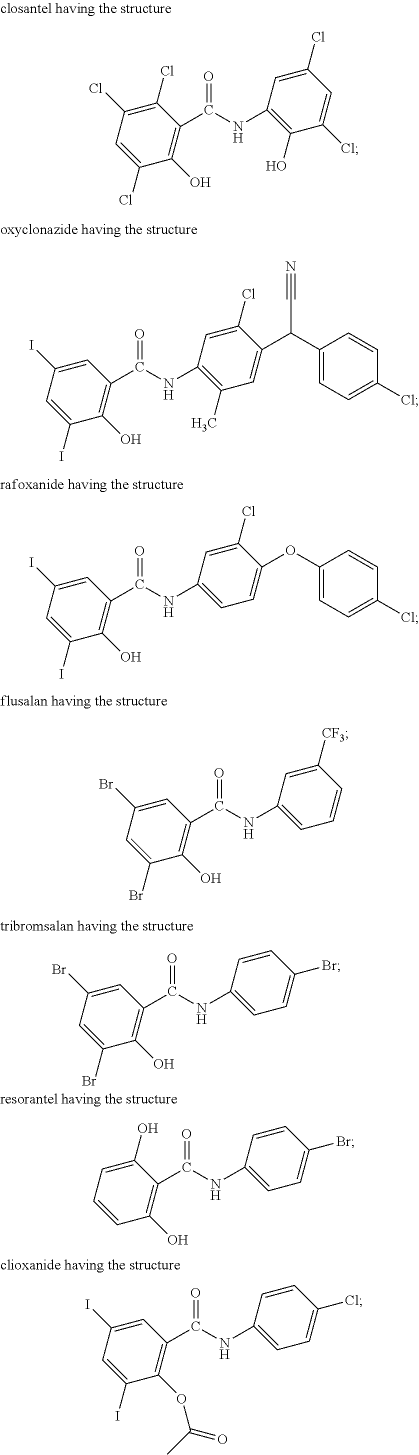

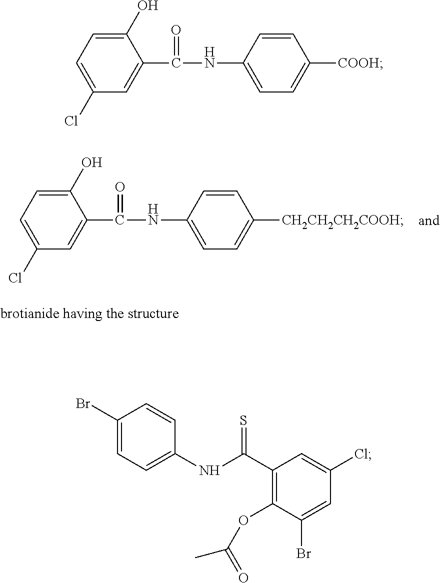

or an ester form thereof or a pharmaceutically acceptable salt thereof.

Alternatively the salicylanilide compound is nitazoxanide (2-acetyloxy-N-(5-nitro-2-thiazolyl)benzamide) or a pharmaceutically acceptable salt thereof.

In an example, the efflux pump inhibitor compound is an inhibitor of a Gram negative bacterium efflux pump, e.g. a homologue of the E. coli AcrAB-TolC efflux pump.

In an example, the efflux pump inhibitor is phenylalanine-arginine β-napthylamide (PAβN) or 2-3 dibromomaleimide.

In another example, the mole ratio of salicylamide compound to efflux pump inhibitor compound is from about 1:500 to about 1:7.

### BRIEF DESCRIPTION OF THE FIGURES

FIG. 1 shows that deletion of the tolC gene greatly sensitizes E. coli to niclosamide. Overnight cultures of E. coli 7KO or 7KOΔtolC strains are used to inoculate fresh aliquots of LB media which are then incubated at 30° C., 200 rpm for 2.5 h. 40 μL aliquots of each culture are subsequently added to 40 μL of LB media containing 2× the desired final niclosamide concentration (i.e., resulting in a final 2-fold dilution series from 5 μM down to 20 nM) or a 0 μM control in a 384-well microplate. The plate is incubated at 30° C., 200 rpm for 4 h. Culture turbidity is monitored by optical density at 600 nm in order to calculate percentage growth relative to the 0 μM control for each strain. Data are the mean of at least two independent experiments±SEM.

FIG. 2 shows that the TolC inhibitor PaβN is able to sensitise E. coli to niclosamide. Overnight cultures of E. coli 7KO are used to inoculate fresh aliquots of LB media which are then incubated at 30° C., 200 rpm for 2.5 h. 40 μL aliquots of each culture are subsequently added to 40 μL of LB media containing 2× the desired final niclosamide concentration (i.e., resulting in a final 2-fold dilution series from 10 μM down to 160 nM) or a 0 μM control, as well as either 0 μM, 25 μM or 50 μM PaβN in a 384-well microplate. An overnight culture of 7KOΔtolC is treated in the same manner, but without addition of PaβN. The 384 well plates are incubated at 30° C., 200 rpm for 4 h. Culture turbidity is monitored by optical density at 600 nm in order to calculate percentage growth relative to the 0 μM niclosamide control for each series. Data are the average of three replicates±1 standard deviation.

FIG. 3 shows relative sensitivity of E. coli strains 7KO and DH5a to niclosamide challenge. Overnight cultures of E. coli 7KO or DH5a are used to inoculate fresh aliquots of LB media which are incubated at 30° C., 200 rpm for 2.5 h. 40 μL aliquots of each culture are subsequently added to 40 μL of LB media containing 2× the desired final niclosamide concentration (i.e., resulting in a final 2-fold dilution series from 40 μM down to 20 nM) or a 0 μM control in a 384-well microplate. The plate is incubated at 30° C., 200 rpm for 4 h. Culture turbidity is monitored by optical density at 600 nm in order to calculate percentage growth relative to the 0 μM control for each strain. Data are the mean of at least two independent experiments±SEM.

FIG. 4 shows niclosamide growth inhibition of E. coli 7KOΔtolC strains over-expressing different native nitroreductase candidates. Overnight cultures of E. coli 7KOΔtolC strains over-expressing different nitroreductase candidate genes as indicated are used to inoculate fresh aliquots of LB media, which are incubated at 30° C., 200 rpm for 2.5 h. 40 μL aliquots of each culture are subsequently added to 40 μL of LB media containing 2× the desired final niclosamide concentration (i.e., resulting in a final 2-fold dilution series from 4 μM down to 16 nM) or a 0 μM control in a 384-well microplate. The plate is incubated at 30° C., 200 rpm for 4 h. Culture turbidity is monitored by optical density at 600 nm in order to calculate percentage growth relative to the 0 μM control for each strain. Data are the mean of at least three independent experiments±SEM.

FIG. 5 shows the abilities of different nitroreductase candidates to defend against challenge with 2.5 μM niclosamide. Overnight cultures of oxidoreductase-overexpressing E. coli 7KOΔtolC strains (origin of each oxidoreductase and nomenclature as described in WO 2012/008860 and Prosser, G. A., Copp, J. N., Mowday, A. M., Guise, C. P., Syddall, S. P., Williams, E. M., Horvat, C. N., Swe, P. S., Ashoorzadeh, A., Denny, W. A., Smaill, J. B., Patterson, A. V. and Ackerley, D. F. (2013). Creation and screening of a multi-family bacterial oxidoreductase library to discover novel nitroreductases that efficiently activate the bioreductive prodrugs CB1954 and PR-104A. Biochemical Pharmacology 85:1091-1103.) are used to inoculate fresh aliquots of assay media (LB media supplemented with 100 μg·mL⁻¹ ampicillin, 0.4% w/v glucose and 50 μM IPTG) which are subsequently incubated at 30° C., 200 rpm for 2.5 h. 40 μL aliquots of each culture are then added to 40 μL aliquots of assay media containing 2× the desired final niclosamide concentration (5 μM) or a media only control in a 384-well microplate. The plate is then incubated at 30° C., 200 rpm for 4 h. Culture turbidity is monitored by optical density at 600 nm in order to calculate percentage growth inhibition relative to the 0 μM control (i.e. 100% growth) for each strain. Data are the mean of three independent experiments±SEM.

FIG. 6 shows niclosamide pre-selection strongly enriches for functional nitroreductases able to bioreductively activate the nitro-prodrug antibiotic metronidazole. A variant gene library for E. coli nfsA is created by codon randomisation at seven active site codon positions, cloned into plasmid pUCX, and transformed into E. coli SOS-R4 cells. 57 clones are randomly selected from either A. LB agar; or B. LB agar containing 500 nM niclosamide. Each of the 57 selected clones (named according to position on a standard 96 well plate) is then tested for growth inhibition in the presence of 50 μM metronidazole (structure inset), with larger values indicating a higher level of growth inhibition, and hence a higher level of metronidazole activation by that clone. Also included on each plate are a wild type nfsA (nfsA_Ec), empty plasmid (pUCX), and media-only control (for reference purposes only). Substantially greater numbers of metronidazole-active clones are present in the niclosamide pre-selected cohort. Growth inhibition data are the mean of three independent experiments±SEM.

FIG. 7 shows niclosamide pre-selection strongly enriches for functional nitroreductases able to bioreductively activate the nitro-prodrug antibiotic tinidazole. A variant gene library for E. coli nfsA is created by codon randomisation at seven active site codon positions, cloned into plasmid pUCX, and transformed into E. coli SOS-R4 cells. 57 clones are randomly selected from either A. LB agar; or B. LB agar containing 500 nM niclosamide. Each of the 57 selected clones (named according to position on a standard 96 well plate) is then tested for growth inhibition in the presence of 50 μM tinidazole (structure inset), with larger values indicating a higher level of growth inhibition, and hence a higher level of tinidazole activation by that clone. Also included on each plate are a wild type nfsA (nfsA_Ec), empty plasmid (pUCX), and media-only control (for reference purposes only). Substantially greater numbers of tinidazole-active clones are present in the niclosamide pre-selected cohort. Growth inhibition data are the mean of three independent experiments±SEM.

FIG. 8 shows a heatmap of niclosamide/PAβN synergy against 3-lactam resistant Klebsiella pneumoniae. This Figure shows percentage growth of β-lactam resistant Klebsiella pneumonia (NZ isolate NIL 05/26) in LB amended with 0.1 M MgSO₄ and niclosamide and PAβN as indicated, relative to unchallenged control. Data are the mean of three independent replicates.

FIG. 9 shows a heatmap of niclosamide/PAβN synergy against 3-lactam resistant E. coli. This Figure shows percentage growth of β-lactam resistant E. coli (NZ isolate ARL06/624) in LB amended with 0.1 M MgSO₄ and niclosamide and PAβN as indicated, relative to unchallenged control. Data are the mean of three independent replicates.

FIG. 10 shows a heatmap of niclosamide/PAβN synergy against ceftazidime/piperacillin resistant Pseudomonas aeruginosa. This Figure shows percentage growth of ceftazidime/piperacillin resistant Pseudomonas aeruginosa (NZ isolate AR 00/537) in LB amended with 0.1 M MgSO₄ and niclosamide and PAβN as indicated, relative to unchallenged control. Data are the mean of three independent replicates.

FIG. 11 shows a heatmap of niclosamide/PAβN synergy against ceftazidime/ciprofloxacin/colistin/meropenem/piperacillin/tobramycin resistant Burkholderia multivorans. This Figure shows percentage growth of ceftazidime/ciprofloxacin/colistin/meropenem/piperacillin/tobramycin resistant Burkholderia multivorans (NZ isolate ARL03/452) in LB amended with 0.1 M MgSO₄ and niclosamide and PAβN as indicated, relative to unchallenged control. Data are the mean of three independent replicates.

FIG. 12 shows a heatmap of niclosamide/PAβN synergy against E. coli lab strain W3110. This Figure shows percentage growth of E. coli lab strain W3110 in LB amended with 0.1 M MgSO₄ and niclosamide and PAβN as indicated, relative to unchallenged control (with data from 7KOΔtolC niclosamide-only control in second left-most column). Data are the mean of three independent replicates.

FIG. 13 shows a heatmap of niclosamide/PAβN synergy against P. aeruginosa lab strain PAO1. This Figure shows percentage growth of P. aeruginosa lab strain PAO1 in LB amended with 0.1 M MgSO₄ and niclosamide and PAβN as indicated, relative to unchallenged control. Data are the mean of three independent replicates.

FIG. 14 shows a heatmap of niclosamide/PAβN synergy against a field isolate of virulent Pseudomonas syringae pv. actinidiae (Psa-V). This Figure shows percentage growth of Psa-V (Landcare isolate ICMP 18800) in LB amended with 0.1 M MgSO₄ and niclosamide and PAβN as indicated, relative to unchallenged control. Data are the mean of three independent replicates.

FIG. 15 shows the relative sensitivity of E. coli strain 7KOΔtolC to niclosamide and 2-chloro-4-nitroaniline. Overnight cultures of E. coli 7KOΔtolC are used to inoculate fresh aliquots of LB media which are incubated at 30° C., 200 rpm for 2.5 h. 40 μL aliquots of each culture are then added to 40 μL aliquots of LB media in a 384-well microplate which contained 2× the desired final concentration of niclosamide or 2-chloro-4-nitroaniline (i.e., resulting in a final 2-fold dilution series from 10 μM down to 160 nM for each compound), or a 0 μM media only control. The plate is incubated at 30° C., 200 rpm for 4 h. Culture turbidity is monitored by optical density at 600 nm in order to calculate percentage growth relative to the 0 μM control for each strain. Data are the mean of at least two independent experiments±SEM.

FIG. 16 shows nitazoxanide growth inhibition of E. coli 7KOΔtolC strains over-expressing different native nitroreductase candidates. Overnight cultures of E. coli 7KOΔtolC (DE3) strains overexpressing either E. coli NfsA (NfsA_Ec), E. coli NfsB (NfsB_Ec) or containing a plasmid only (pET28) control are used to inoculate fresh aliquots of assay media (LB media supplemented with 50 μg·mL⁻¹ kanamycin and 50 μM IPTG) which are incubated at 30° C., 200 rpm for 2.5 h. 40 μL aliquots of each culture are then added to 40 μL aliquots of assay media containing 2× the desired final concentration of nitazoxanide (i.e., resulting in a final 2-fold dilution series from 20 down to 2.5 μM) or a 0 μM media only control. The plate is then incubated at 30° C., 200 rpm for 4 h. Culture turbidity is monitored by optical density at 600 nm in order to calculate percentage growth relative to the 0 μM control for each strain. Data are the mean of at least two independent experiments±SEM. The structure of nitazoxanide is inset.

FIG. 17 shows primers used for in-frame deletion of candidate nitroreductase genes and tolC gene from the E. coli chromosome. Gene knockouts are performed by in frame deletion using Red recombinase (Datsenko, K. A. and Wanner, B. L. (2000). One-step inactivation of chromosomal genes in Escherichia coli K-12 using PCR products. Proc Natl Acad Sci USA 97:6640-6645.). Briefly, plasmid pKD4 is used as a template to PCR-amplify a kanamycin resistance gene flanked either side by flp-recombinase recognition sites. Primers for amplification contain 15-20 bp of sequence at the 3′ end for priming and amplification from pKD4. The remaining N40 bp at the 5′ end of the primers are homologous to either end of the genomic region targeted for deletion. In order to improve knock-out efficiency in certain cases, the genomic homologous regions at each end of the PCR-amplified kanamycin cassette are lengthened via a second PCR, using the first PCR product as template and knock-out extension primers for amplification. Primers are named according to the gene to be knocked out (KO) with the suffix FW indicating a forward primer, RV a reverse primer, and EXT an extension primer (e.g., NFSA_KO_FW is the forward primer for knockout of gene nfsA).

FIG. 18 shows a 384 well plate format for “heatmap” measurement of growth inhibition across a range of niclosamide and PAβN concentrations, in quadruplicate. The final niclosamide and PAβN concentrations for each well are shown. In this example, the culture media for row H, columns 1 and 3 would contain 40 μM niclosamide and 150 μM PAβN to allow for a subsequent 1 in 2 dilution with bacterial culture.

FIG. 19 shows a heatmap of effect of combined or individual Niclosamide and PAβN treatments on methicillin resistant Staphylococcus aureus (MRSA). This Figure shows percentage growth of methicillin resistant Staphylococcus aureus (MRSA; ATCC43300) in LB amended with 0.1 M MgSO₄ and niclosamide and PAβN as indicated, relative to unchallenged control. Data are the mean of three independent replicates.

FIG. 20 shows that Gram positive bacteria are directly sensitive to niclosamide, without the need for co-administration of a TolC inhibitor. Shown are % growth inhibition curves for the Gram positive strains S. aureus ATCC 43300, L. welshimeri ATCC 35897, and B. thuringiensis P.1.IPS-80 serovar israelensis across a range of niclosamide concentrations, relative to an unchallenged control for each strain. Data are the mean of two independent replicates (using duplicate technical replicates for each independent experiment) and error bars indicate standard error of the mean. The IC₅₀ values calculated from these curves are presented in Table 1.

FIG. 21 shows that Gram positive bacteria are directly sensitive to nitazoxanide, without the need for co-administration of a TolC inhibitor. Shown are % growth inhibition curves for the Gram positive strains S. aureus ATCC 43300, L. welshimeri ATCC 35897, and B. thuringiensis P.1.IPS-80 serovar israelensis across a range of nitazoxanide concentrations, relative to an unchallenged control for each strain. Data are the mean of two technical replicates. The IC₅₀ values calculated from these curves are presented in Table 1.

### DETAILED DESCRIPTION

The present invention is predicated on the surprising and unexpected discovery that salicylamide compounds display direct growth inhibition of Gram positive bacteria. Accordingly, the present invention is concerned with compositions and methods effective in the prevention or treatment of bacterial infections, and/or in the prevention, reduction or elimination of biofilm formation involving salicylamide compounds.

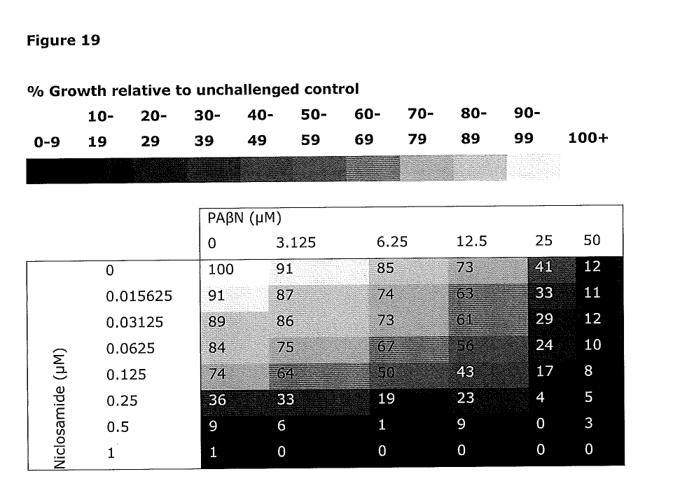

A clinical isolate of the Gram positive bacterium methicillin resistant Staphylococcus aureus (MRSA; ATCC43300) was tested. Refer to FIG. 19. Surprisingly, this strain is sensitive to micromolar levels of PaβN as well as being sensitive to niclosamide at nanomolar concentrations. Consistent with Gram positive bacteria lacking TolC efflux mechanisms, it appears that niclosamide is effective in the absence of PaβN; and that the combined effects of niclosamide and PaβN treatments are additive rather than synergistic (FIG. 19).

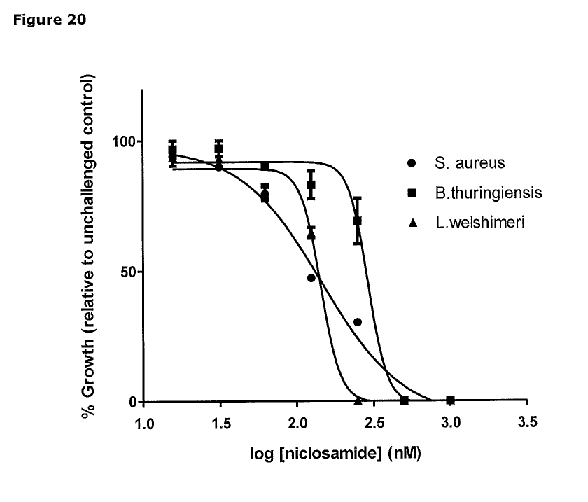

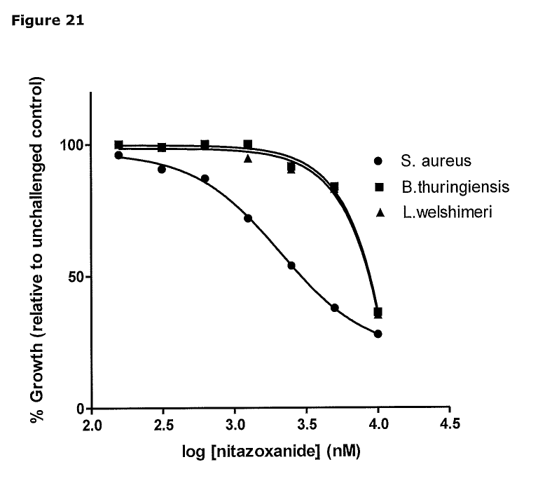

These data prompted the applicants to further investigate the direct growth inhibitory effects of salicylamide compounds on Gram positive bacteria. With reference to FIGS. 20 and 21, when read in conjunction with the data presented in Table 2 (refer to the Examples which follow), niclosamide and nitazoxanide demonstrate direct growth inhibition activity against Staphylococcus aureus, Listeria welshimeri and Bacillus thuringiensis with IC₅₀ values in the nM range. This represents an important finding and demonstrates the utility for salicylamide compounds on the growth inhibition of Gram positive bacteria responsible for bacterial infection and/or which form (part of) a bacterial biofilm.

Accordingly, in one aspect of the present invention there is provided a salicylamide compound for use in treating a bacterial infection in a patient, wherein the bacteria causing infection comprises Gram positive bacteria.

In another aspect the present invention provides a salicylamide compound for use in preventing, reducing or eliminating formation of a bacterial biofilm, wherein the bacteria causing biofilm formation comprises Gram positive bacteria.

The salicylamide compound may be formulated as a pharmaceutical or biological composition, together with an acceptable excipient or carrier. The salicylamide compound may also be formulated as a pharmaceutical salt.

The invention further provides methods and uses comprising the salicylamide compounds according to the present invention for treating or preventing a bacterial infection comprising Gram positive bacteria, or for preventing, reducing or eliminating a biofilm formation, wherein the biofilm comprises Gram positive bacteria.

In this specification, the term “patient” my include, for example, a patient with an infection, or predisposed to risk of infection, as well as a medical practitioner administering one or more actives for the treatment of a patient with an infection or predisposed to risk of acquiring an infection. For example, the present invention may provide a biological composition comprising a salicylamide compound, optionally in conjunction with an efflux pump inhibitor, formulated as a hand-sanitising agent for use by surgeons prior to surgery. Additionally or alternatively, for example, the present invention may provide a pharmaceutical composition comprising a salicylamide compound, optionally in conjunction with an efflux pump inhibitor, for administration to a patient during surgery, either to treat a patient having an infection or to prevent a patient from acquiring an infection by one or more bacteria during surgery.

The present invention is also predicated on the surprising and unexpected discovery that growth inhibition of Gram negative bacteria may be achieved using a salicylamide compound in combination with an efflux pump inhibitor.

The combinations and compositions of the present invention are therefore useful for the treatment or prevention of infection, particularly in humans, and for the prevention, reduction or elimination of biofilm formation, among other applications.

In some examples, the mole ratio of salicylamide compound to efflux pump inhibitor compound is from about 1:500 to about 1:7, e.g. about 1:400 to about 1:7, e.g. about 1:350 to about 1:7, e.g. about 1:300 to about 1:7, e.g. about 1:250 to about 1:7, e.g. about 1:200 to about 1:7, e.g. about 1:150 to about 1:7, e.g. about 1:100 to about 1:7, e.g. about 1:50 to about 1:7, e.g. about 1:20 to about 1:7, e.g. about 1:10 to about 1:7.

The applicants have surprisingly found that niclosamide is toxic to E. coli SOS-R2 cells that are not over-expressing an active nitroreductase, whereas active nitroreductases are found to enhance growth of SOS-R2 in the presence of niclosamide. However, in a different E. coli host strain (“6KO”, a derivative of E. coli W3110 that has six endogenous nitroreductase candidate genes knocked out) niclosamide is no longer found to be toxic, even if the strain is not over-expressing an active nitroreductase. Without wishing to be bound by theory, the applicants hypothesise that a key difference between the niclosamide-sensitive SOS-R2 and niclosamide-resistant 6KO strains is that the former carry a deletion of the tolC gene, which encodes an efflux pump capable of exporting numerous xenobiotic and other compounds directly across both cell membranes of Gram negative bacterial cells.

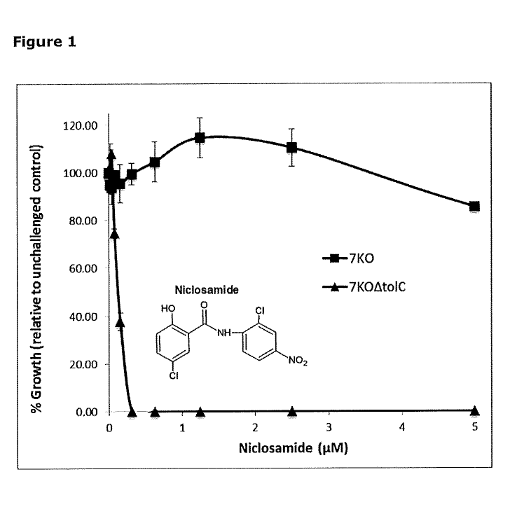

FIG. 1 shows that deletion of the tolC gene sensitises E. coli to niclosamide. This experiment measures the relative sensitivities to niclosamide of two otherwise isogenic E. coli strains, one with an intact tolC gene and the other carrying an in-frame deletion of tolC. To avoid any potential confounding effects due to nitroreductase activity, the base strain selected for this study is 7KO—E. coli W3110 carrying in-frame deletions of five verified nitroreductase genes (nfsA, nfsB, azoR, nemA, mdaB) and two suspected nitroreductases (yieF, ycaK). The endogenous tolC gene is deleted in-frame from this strain using the Red recombinase method of Datsenko and Warner (Datsenko, K. A. and Wanner, B. L. (2000). One-step inactivation of chromosomal genes in Escherichia coli K-12 using PCR products. Proc Natl Acad Sci USA 97:6640-6645.) to give strain 7KOΔtolC.

When growth of replicate 7KO and 7KOΔtolC cultures is compared across a 2-fold dilution series of niclosamide (from 5 μM down to 20 nM), it is apparent that the tolC mutation confers extreme sensitivity to niclosamide (FIG. 1). From these data the niclosamide IC₅₀ (concentration of niclosamide at which growth of a niclosamide challenged replicate is predicted to be 50% that of the unchallenged control) is calculated to be 120 nM for 7KOΔtolC, but for 7KO the IC₅₀ cannot be calculated (i.e., is substantially greater than 5 μM).

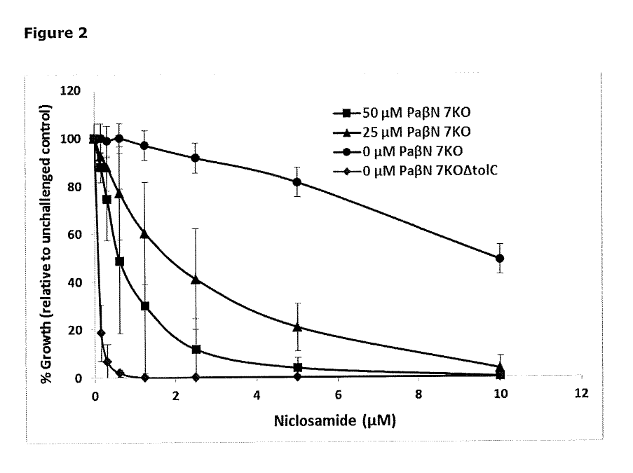

As the tolC gene deletion is constructed in-frame, it is unlikely that the niclosamide sensitivity phenotype is a consequence of a polar effect (i.e., due to an influence of the deleted DNA region on neighbouring genes, rather than the tolC mutation itself). This is confirmed by the experiment shown in FIG. 2, which demonstrates that chemical inhibition of the TolC efflux pump also sensitises E. coli to niclosamide. Replica cultures of E. coli 7KO are grown across a 2-fold dilution series of niclosamide (from 10 μM down to 156 nM) in the presence of either 0 μM, 25 μM or 50 μM of phenylalanine-arginine 3-napthylamide (PAβN), a chemical inhibitor of TolC efflux pumps (Lomovskaya, O., Warren, M. S., Lee, A., Galazzo, J., Fronko, R., Lee, M., Blais, J., Cho, D., Chamberland, S., Renau, T., Leger, R., Hecker, S., Watkins, W., Hoshino, K., Ishida, H. and Lee, V. J. (2001). Identification and Characterization of Inhibitors of Multidrug Resistance Efflux Pumps in Pseudomonas aeruginosa: Novel Agents for Combination Therapy. Antimicrobial Agents and Chemotherapy 45(1): 105-116.). Addition of PAβN is found to promote niclosamide sensitivity in the 7KO strain in a dose-dependent manner, although at the concentrations tested the effect is less than observed for the 7KOΔtolC, where TolC activity is completely eliminated.

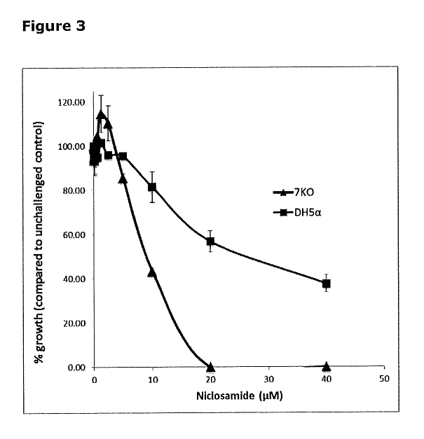

Thus, the applicants demonstrate, by both genetic and chemical means, that TolC is able to defend E. coli against niclosamide. The experiment shown in FIG. 3 compares the ability of nitroreductase enzymes to defend against niclosamide by examining the effect of endogenous nitroreductase enzymes (i.e., expressed from native chromosomal nitroreductase genes naturally found in E. coli) to defend cells containing a functional tolC gene against high level niclosamide challenge. The applicants compared growth of the 7KO strain (carrying deletions of the candidate nitroreductase genes nfsA, nfsB, azoR, nemA, mdaB, yieF, ycaK) to the commercially available cloning strain DH5a, in which all seven candidate nitroreductase genes are intact. When growth of replicate cultures is compared across a 2-fold dilution series of niclosamide (from 40 μM down to 20 nM), it is apparent that DH5a is more resistant to niclosamide than the 7KO strain (FIG. 3).

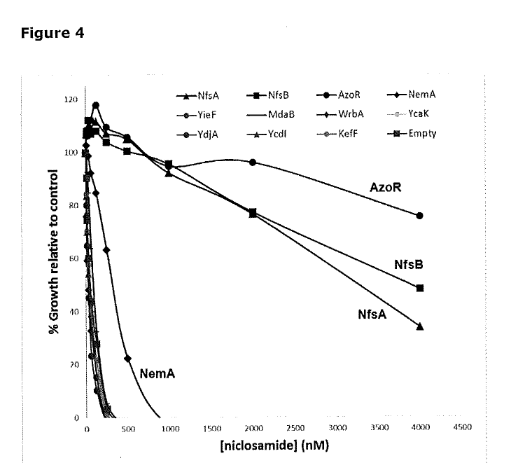

FIG. 4 shows that over-expressed nitroreductase genes can provide high-level defence against niclosamide challenge. This experiment examines whether high-level expression of nitroreductase genes under control of a strong promoter on a multi-copy plasmid provide high level defence to E. coli 7KOΔtolC (i.e., cells that are pre-sensitised to niclosamide because they do not have a TolC-mediated defence system). When growth of replicate cultures over-expressing each nitroreductase candidate from plasmid pUCX (Prosser, G. A., Copp, J. N., Syddall, S. P., Williams, E. M., Smaill, J. B., Wilson, W. R., Patterson, A. V. and Ackerley, D. F. (2010). Discovery and evaluation of Escherichia coli nitroreductases that activate the anti-cancer prodrug CB1954. Biochemical Pharmacology 79: 678-687.) is compared across a 2-fold dilution series of niclosamide (from 4 μM down to 16 nM), it can be seen that the nitroreductases NfsA, NfsB and AzoR are able to provide high level protection against niclosamide (strains over-expressing those enzymes having low micromolar IC₅₀ values for niclosamide), whereas NemA affords a much lower level of protection (IC₅₀=300 nM) (FIG. 4). Another previously validated E. coli nitroreductase, MdaB, has an IC₅₀ of the MdaB over-expressing strain of 83 nM, which is not substantially greater than that of the empty plasmid control (IC₅₀=47 nM).

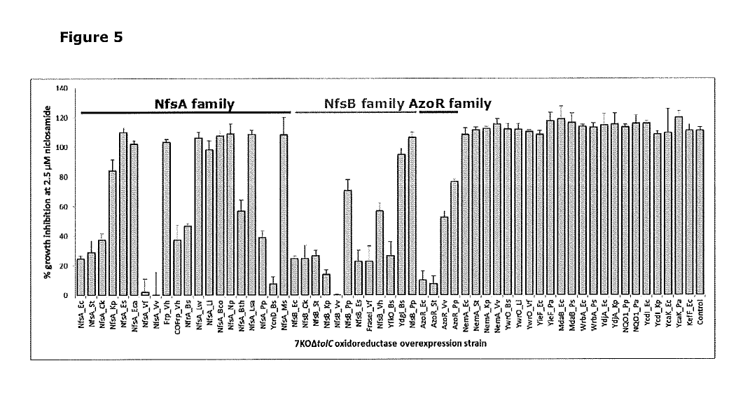

FIG. 5 shows the results from screening of a 58-membered oxidoreductase pUCX library that contains members of 11 different oxidoreductase families (WO 2012/008860; Prosser, G. A., Copp, J. N., Mowday, A. M., Guise, C. P., Syddall, S. P., Williams, E. M., Horvat, C. N., Swe, P. S., Ashoorzadeh, A., Denny, W. A., Smaill, J. B., Patterson, A. V. and Ackerley, D. F. (2013). Creation and screening of a multi-family bacterial oxidoreductase library to discover novel nitroreductases that efficiently activate the bioreductive prodrugs CB1954 and PR-104A. Biochemical Pharmacology 85:1091-1103.) in 7KOΔtolC cells at a single concentration of niclosamide (2.5 μM) to identify nitroreductases that can defend the host cell against high level niclosamide challenge. Members of the NfsA, NfsB and AzoR families are consistently active, whereas no members of any of the other eight oxidoreductase families enable growth of 7KOΔtolC cells at that concentration of niclosamide.

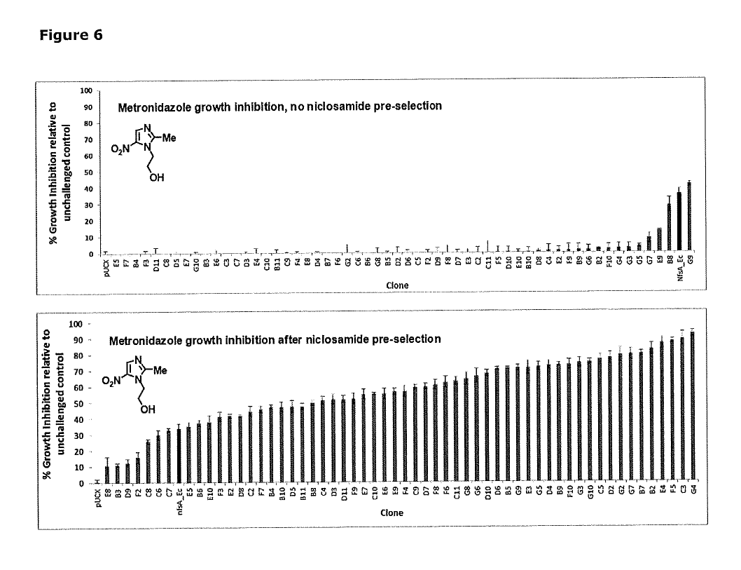

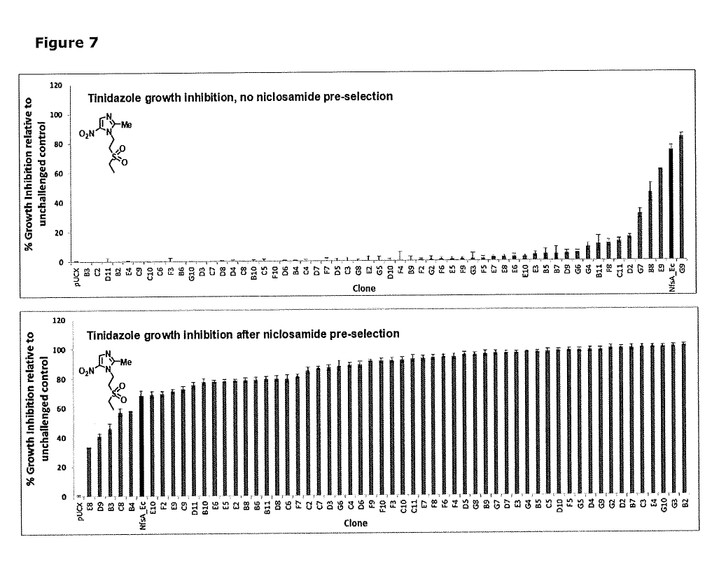

FIGS. 6 and 7 show that niclosamide can be used to pre-select functional nitroreductases from mutated gene libraries expressed in tolC mutant host cells. Nitroreductases have a wide range of potential applications in biotechnology. Of particular interest for environmental applications is the ability of nitroreductase enzymes to catalyze the conversion of toxic xenobiotic pollutants into less toxic forms (Roldán, M.D., Pérez-Reinado, E., Castillo, F. and Moreno-Vivián, C. (2008). Reduction of polynitroaromatic compounds: the bacterial nitroreductases. FEMS Microbiol Rev 32(3):474-500.). Conversely, the conversion of prodrugs into highly cytotoxic forms has applications in medicine (e.g. the anti-cancer strategy gene-directed enzyme prodrug therapy (Schellmann, N., Deckert, P. M., Bachran, D., Fuchs, H. and Bachran C. (2010). Targeted enzyme prodrug therapies. Mini Rev Med Chem. 10: 887-904.) or cell biology (e.g. targeted tissue ablation in transgenic model organisms) (Curado Rosenthal, V. D., Maki, D. G., Jamulitrat, S., Medeiros, E. A., Todi, S. K., Gomez, D. Y., Leblebicioglu, H., Abu Khader, I., Miranda Novales, M. G., Berba, R., Ramirez Wong, F. M., Barkat, A., Pino, O. R., Dueias, L., Mitrev, Z., Bijie, H., Gurskis, V., Kanj, S. S., Mapp, T., Hidalgo, R. F., Ben Jaballah, N., Raka, L., Gikas, A., Ahmed, A., Thu, L. T. A. and Guzman Siritt, M. E. (2010). International Nosocomial Infection Control Consortium (INICC) report, data summary for 2003-2008, issued June 2009. American Journal of Infection Control 38(2): 95-104). Nitroreductases are also of interest for biocatalysis, that is, reduction of nitro groups during chemical syntheses, e.g. pharmaceutical manufacture. In all of these scenarios, nitroreductases are generally being applied for reduction of non-physiological substrates, relying on their typical substrate promiscuity (Roldan et al., 2008). Thus, it is likely that native nitroreductase enzymes will not be particularly efficient with the desired substrate, and that their starting level of promiscuous activity might be able to be improved substantially by engineering strategies such as directed evolution.

Typically in directed evolution the more a target gene is mutated, the more likely it is to become inactivated. Thus, achieving an evolved enzyme that contains a large number of mutations typically requires a very large number of clones to have been screened for improved activity. However, many screens for desirable activities do not have a very high throughput capability.

Without wishing to be bound by theory, the applicants hypothesise that niclosamide pre-selection of a substantially mutated nitroreductase library would greatly enrich for functional nitroreductases, enabling low throughput screening approaches such as growth inhibition assays to recover variants with enhanced activity for particular substrates. A mutant gene library is synthesised (by GenScript), based on E. coli nfsA, with the codons for seven active site residues partially (NDT codon set) or fully (NNK codon set) randomized. In all, the library contains N95 million gene variants, the vast majority of which are expected to encode inactive nitroreductases. This library is transformed into E. coli SOS-R4 cells (which contain knockouts of the nfsA, nfsB, azoR, nemA and tolC genes, as well as a plasmid-borne SOS-regulated GFP gene) (Copp, J. N., Williams, E. M., Rich, M. H., Patterson, A. V., Smaill, J. B. and Ackerley, D. F. (2014). Toward a high-throughput screening platform for directed evolution of enzymes that activate genotoxic prodrugs. Protein Eng Des Sel. 27(10):399-403.) and a range of dilutions is plated onto replica LB agar plates, either unamended or amended with 500 nM niclosamide. 57 colonies are randomly selected from an unamended LB agar plate and are inoculated, together with empty plasmid and wild type NfsA control colonies, and a cell-free control, into LB in the 60 innermost wells of a 96-well plate. The procedure is repeated, into a different 96 well plate using 57 colonies randomly selected from a niclosamide-amended LB agar plate (plus the same three controls). Following this, growth inhibition assays are employed to measure how many wells per plate contained clones expressing enzyme variants that are active with the nitro-prodrug antibiotics metronidazole (FIG. 6A, 6B) or tinidazole (FIG. 7A, 7B).

In the absence of niclosamide pre-selection, only one of the 57 randomly selected clones is found to express a nitroreductase variant that is more active than wild type NfsA with metronidazole (FIG. 6A) or tinidazole (FIG. 7A). However, following niclosamide pre-selection, 50 out of 57 clones are more active than wild type NfsA with metronidazole (FIG. 6B) and 52 out of 57 clones are more active than wild type NfsA with tinidazole (FIG. 7B). These data indicate that niclosamide pre-selection can provide a powerful enrichment for genes encoding functional nitroreductase enzyme variants from a mutant gene library.

Accordingly, in one aspect the present invention provides a screening method to identify novel nitroreductase enzymes, the method comprising the steps of:

(i) performing a targeted or random mutagenesis of an existing nitroreductase gene thereby producing a nitroreductase variant gene library; and
(ii) transforming the variant gene library into Gram negative bacteria in which the tolC gene has been deleted or the tolC expression product has been inhibited and culturing the cells so that gene variant is expressed; and
(iii) administering at least one salicylamide compound to the transformed bacterial cells; and
(iv) screening for cells which lack susceptibility to salicylamide toxicity thereby identifying cells which express a novel form of the nitroreductase enzyme; and
(v) optionally purifying the nitroreductase enzyme.

In an example, the endogenous nitroreductase genes of the Gram negative bacteria have been knocked out or nitroreductase activity in the Gram negative bacteria has been reduced or eliminated.

In yet another aspect the present invention provides a screening method to identify novel nitroreductase enzymes from a preparation of environmentally sourced DNA, the method comprising the steps of:

(i) generating a bacterial gene library from that environmentally sourced DNA; and
(ii) transforming the gene library into Gram negative bacteria in which the tolC gene has been deleted or the tolC expression product has been inhibited and culturing the cells so that gene library is expressed; and
(iii) administering at least one salicylamide compound to the transformed bacterial cells; and
(iv) screening for cells which lack susceptibility to salicylamide thereby identifying cells which express a novel form of the nitroreductase enzyme; and
optionally purifying the nitroreductase enzyme.

In an example, the endogenous nitroreductase genes of the Gram negative bacteria have been knocked out or nitroreductase activity in the Gram negative bacteria has been reduced or eliminated.

In another example the environmentally sourced DNA is sourced from soil.

Also contemplated by the present invention is a method to screen for novel TolC inhibitors, based on a screening assay involving bacteria susceptible to salicylamide toxicity, for example, niclosamide and niclosamide analogs.

Accordingly, in yet another aspect the present invention provides a screening method to identify novel inhibitors of TolC, the method comprising the steps of:

(i) culturing Gram negative bacteria which express TolC in the presence of at least one salicylamide compound and a candidate inhibitor of TolC; and
(ii) screening for cells which are susceptible to salicylamide toxicity
thereby identifying novel inhibitors of TolC.

The applicants have also shown that niclosamide and a chemical inhibitor of TolC surprisingly provide a synergistic antibacterial combination effective against a wide range of Gram negative bacteria including multi-drug resistant clinical isolates. Advantageously, niclosamide is known to be tolerated in humans at high doses, and the applicants' work also demonstrates that it is an effective antibiotic against Gram negative bacteria, applied in combination with a chemical inhibitor such as PaβN that has broad spectrum activity against Gram negative TolC efflux pumps. The applicants' work uses growth inhibition assays to test the combined effects of niclosamide and PaβN treatment on a range of drug-resistant clinical isolates of bacterial pathogens obtained from the ESR culture collection (http://www.esr.cri.nz/competencies/Health/Pages/nzrcc.aspx) as well as a high virulence field isolate of the kiwifruit pathogen Pseudomonas syringae pv. actinidiae (Psa-V) (Landcare isolate ICMP 18800) and laboratory strains of E. coli W3110 and Pseudomonas aeruginosa PAO1 from the applicants' existing stocks.

Accordingly, in one aspect the present invention provides combination products comprising at least one salicylamide compound and at least one efflux pump inhibitor.

In another aspect the present invention provides a synergistic combination of at least one salicylamide compound and at least one efflux pump inhibitor compound.

In another aspect the present invention provides a composition comprising at least one salicylamide compound and at least one efflux pump inhibitor compound. In an example the composition comprises synergistically effective amounts of the salicylamide compound and the efflux pump inhibitor compound.

In a further aspect the present invention provides a pharmaceutical composition comprising at least one salicylamide compound and at least one efflux pump inhibitor compound, together with a pharmaceutically acceptable excipient or salt.

In one example according to the present invention, the salicylamide compound is niclosamide or a niclosamide analog. Examples of niclosamide analogs are listed below.

Examples of suitable efflux pumps for use in the combination products and compositions of the present invention are listed below. In certain embodiments of the present invention the efflux pump inhibitor is a TolC efflux pump inhibitor. An example of a TolC efflux pump inhibitor includes, but is not limited to, PaβN and 2,3-dibromomaleide.

The testing format is a two dimensional 384 well plate assay where replica cultures of each test strain are challenged with increasing concentrations of PaβN on the horizontal axis, and increasing concentrations of niclosamide on the vertical axis (each prepared as a two-fold dilution series, from right-to-left for PaβN and bottom-to-top for niclosamide). Each 384 well plate is divided into four quadrants, such that the top left well in each quadrant contains neither PaβN nor niclosamide, whereas the bottom right well in each quadrant contains the highest concentration of each of PaβN and niclosamide. Each test strain is then evaluated in quadruplicate on each plate.

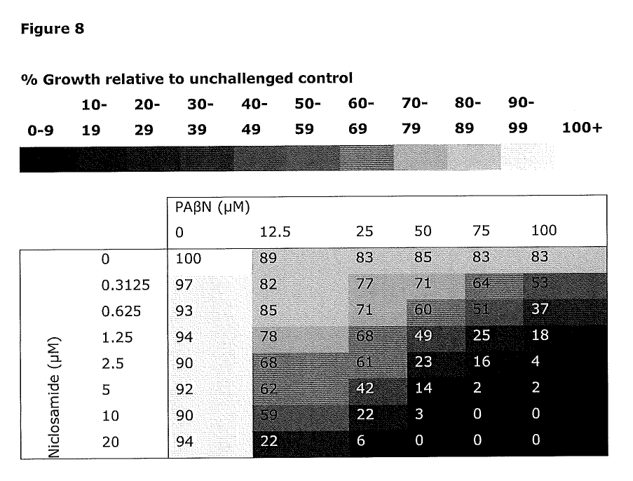

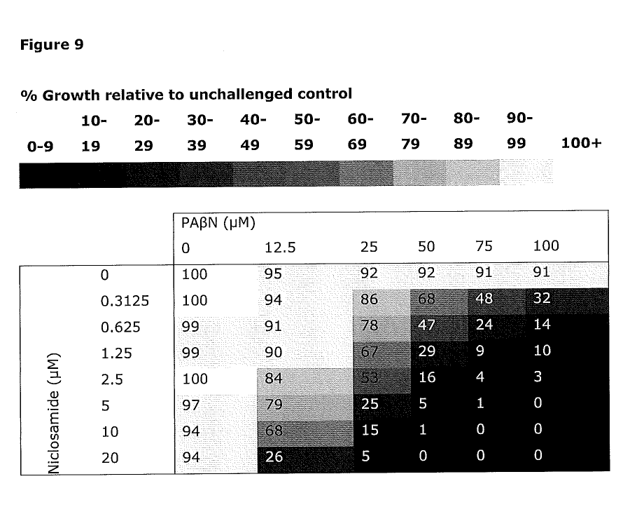

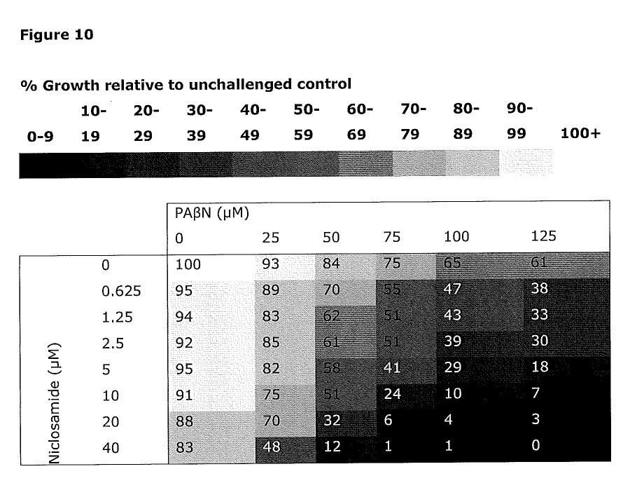

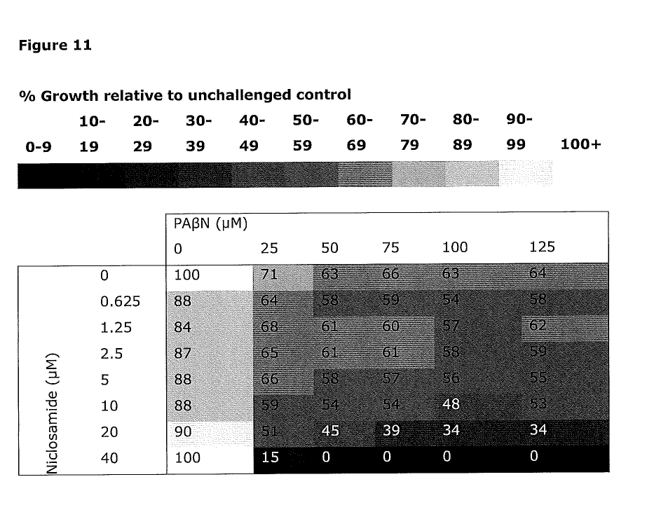

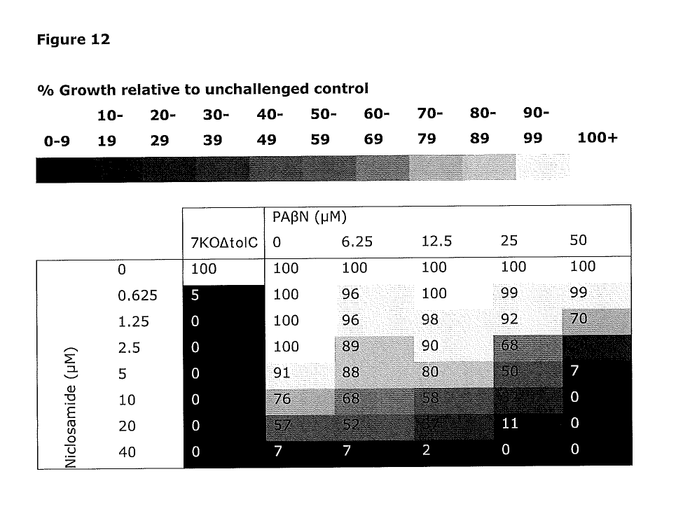

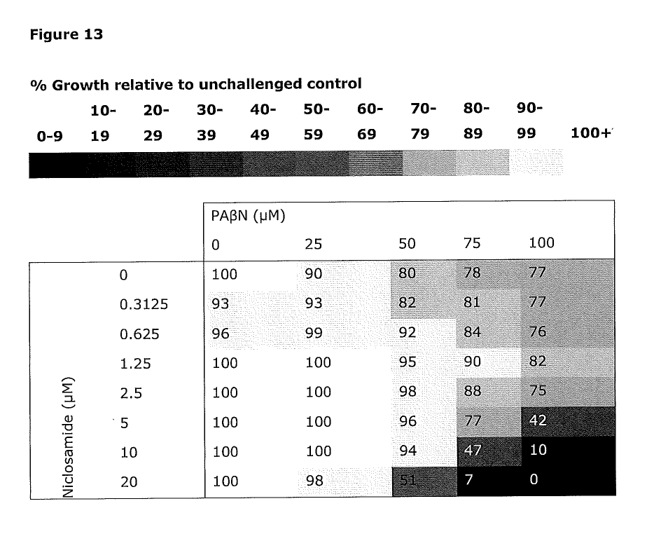

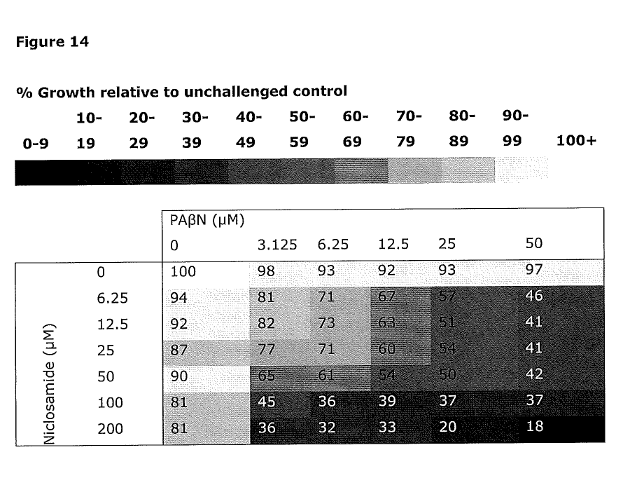

Gram negative strains tested in this format include:
β-lactam resistant Klebsiella pneumonia (NZ isolate NIL 05/26) (FIG. 8) β-lactam resistant E. coli (NZ isolate ARL06/624) (FIG. 9)
Ceftazidime/piperacillin resistant Pseudomonas aeruginosa (NZ isolate AR 00/537) (FIG. 10)
Ceftazidime/ciprofloxacin/colistin/meropenem/piperacillin/tobramycin resistant Burkholderia multivorans (NZ isolate ARL03/452) (FIG. 11)
E. coli W3110 (wild type) (FIG. 12)
P. aeruginosa PAO1 (wild type) (FIG. 13)
Psa-V (Landcare isolate ICMP 18800) (FIG. 14)

In each case the tested strain is sensitive to PaβN and niclosamide as a synergistic combination. However, none of the clinical isolates are particularly sensitive to niclosamide in isolation (IC₅₀>20 μM in the absence of PaβN; FIGS. 8-11).

The combination products or compositions according to the invention may therefore be used to treat or prevent a bacterial infection in a patient, or may be used to reduce or eliminate formation of a bacterial biofilm, wherein the bacteria causing infection or biofilm formation comprise Gram negative bacteria.

Accordingly, in another aspect the present invention provides use of a combination of at least one salicylamide compound and at least one efflux pump inhibitor compound or a composition comprising at least one salicylamide compound and at least one efflux pump inhibitor compound as a medicament.

In another aspect the present invention provides a combination of at least one salicylamide compound and at least one efflux pump inhibitor compound for use in the preparation of a pharmaceutical composition.

In yet another aspect the present invention provides the use of a combination of at least one salicylamide compound and at least one efflux pump inhibitor compound for treating or preventing a bacterial infection in a patient, wherein the bacteria causing infection comprise Gram negative bacteria.

In another aspect the present invention provides the use of a pharmaceutical composition comprising a pharmaceutically effective amount of at least one salicylamide compound and at least one efflux pump inhibitor compound for treating or preventing a bacterial infection in a patient, wherein the bacteria causing infection comprise Gram negative bacteria.

In a further aspect the present invention provides an anti-bacterial agent comprising at least one salicylamide compound and at least one efflux pump inhibitor compound. The anti-bacterial agent may be used to treat or prevent a bacterial infection in a patient, or it may be used to reduce or eliminate formation of a bacterial biofilm, wherein the bacteria causing infection or biofilm formation comprise Gram negative bacteria.

A biofilm has the potential to cause infection in a wound and/or burn or causes an infection on or in an in-dwelling medical device. Alternatively, formation of bacterial biofilms occurs within preparative machinery for the food industry, on packaging used by the food industry, within storage tanks used for water or other liquids, or within machinery at water treatment plants, all of which have the potential to increase the risk of infection arising from human or animal contact with consumable products. Further, the accumulation of bacteria via biofilm formation on surfaces such as hospital beds, bathrooms and doors connecting wards etc also has the ability to expose humans to risk on infection.

Accordingly, the ability to not only treat or prevent a bacterial infection in humans (and animals), but to reduce or eliminate formation of bacterial biofilms is an equally important consideration for use of the combination products and compositions of the invention.

The combinations and compositions of the present invention are also useful for the treatment or prevention of infections in plants, for example bacterial infections caused by Pseudomonas syringae pv. actinidiae (Psa-V) in kiwifruit plants of the genus Actinidia. In an example the combinations and compositions of the present invention exhibit synergistic effects with regard to the treatment or prevention of bacterial infections.

In certain embodiments, the combination products or compositions according to the invention may further comprise one or more bactericidal or bacteriostatic agents. Examples of bactericidal agents include, but are not limited to, beta lactam antibiotics (e.g. penicillin derivatives, cephalosporins, monobactams, carbapenems), vancomycin, daptomycin, fluoroquinolones, metronidazole, nitrofurantoin, co-trimoxazole or telithromycin Examples of bacteriostatic agents include, but are not limited to tetracyclines, macrolides, sulfonamides, lincosamides, oxazolidinone, tigecycline, novobiocin, nitrofurantoin, spectinomycin, trimethoprim, chloramphenicol, ethambutol or clindamycin.

The rise in antibiotic resistance is having a profound impact on the healthcare industry, and the need to provide alternative medicines to combat bacterial infection (i.e. to treat or prevent infection) is growing increasingly important. Accordingly, in another aspect the present invention provides use of at least one salicylamide compound and at least one efflux pump inhibitor compound in the manufacture of a medicament or a combination of at least one salicylamide compound and at least one efflux pump inhibitor compound for use in the manufacture of a medicament.

In another aspect the present invention provides a pharmaceutical composition comprising at least one salicylamide compound and at least one efflux pump inhibitor compound for treating or preventing a bacterial infection in a patient, wherein the bacteria causing infection comprise Gram negative bacteria.

In yet another aspect the present invention provides the use of at least one salicylamide compound and at least one efflux pump inhibitor compound in the manufacture of a medicament for treating or preventing a bacterial infection in a patient, wherein the bacteria causing infection comprise Gram negative bacteria.

In yet another aspect the present invention provides a kit of parts comprising at least one salicylamide compound and at least one efflux pump inhibitor compound in separate unit dosage forms, together with instructions for use. The kits according to the invention could be prescribed and/or administered by healthcare practitioners as a new way of combating infection.

In yet another aspect the present invention provides a method of treating or preventing a bacterial infection, comprising administering, to a patient requiring treatment, at least one salicylamide compound and at least one efflux pump inhibitor compound in amounts sufficient to treat or prevent the bacterial infection in the patient, wherein the bacteria causing infection comprise Gram negative bacteria.

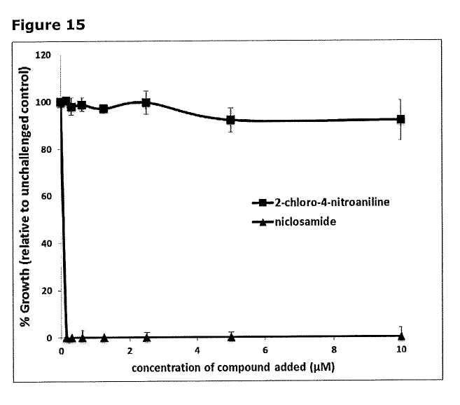

Hydrolysis of niclosamide is predicted to yield 5-chlorosalicyclic acid and 2-chloro-4-nitroaniline. Mutagenicity studies (Espinosa-Aguirre, J. J., Reyes, R. E. and Cortinas de Nava, C. (1991). Mutagenic activity of 2-chloro-4-nitroaniline and 5-chlorosalicylic acid in Salmonella typhimurium: two possible metabolites of niclosamide. Mutation Research Letters 264(3): 139-145.) suggest that 2-chloro-4-nitroaniline is the mutagenic product whereas 5-chlorosalicyclic acid is non-mutagenic. FIG. 15 shows the relative abilities of niclosamide and 2-chloro-4-nitroaniline to inhibit growth of the E. coli strain 7KOΔtolC. 2-chloro-4-nitroaniline is at least three orders of magnitude less toxic than niclosamide, suggesting that this hydrolysed derivative of niclosamide is not the primary antibacterial agent via which niclosamide toxicity is effected.

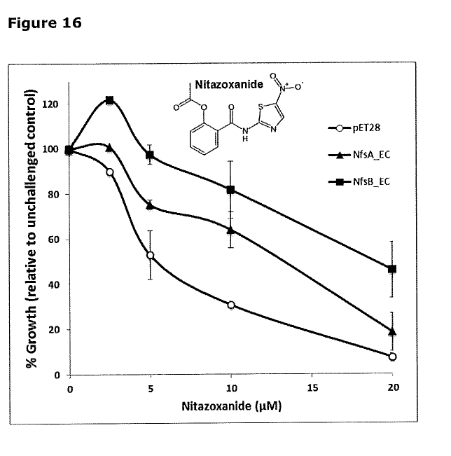

Nitazoxanide (FIG. 16, inset) is a salicylanilide compound that can be used in the combinations and compositions of the invention. Nitazoxanide is the preferred treatment course for Cryptosporidium parvum and Giardia lambia infection (Anderson, V. R. and Curran, M. P. (2007). Nitazoxanide: a review of its use in the treatment of gastrointestinal infections. Drugs. 67(13):1947-1967.). Nitazoxanide disrupts membrane potential and pH homeostasis in Mycobacterium tuberculosis and inhibits pyruvate oxidoreductases in Helicobacter and Campylobacter as well as other anaerobic bacteria and parasites (de Carvalho, L. P. S., Darby, C. M., Rhee, K. Y. and Nathan, C. (2011). Nitazoxanide disrupts membrane potential and intrabacterial pH homeostasis of Mycobacterium tuberculosis. ACS medicinal chemistry letters 2(11): 849-854.). In E. coli 7KOΔtolC (DE3) cells (strain 7KOΔtolC with an integrated ADE3 prophage to allow for inducible expression of genes under T7 RNA polymerase promoter control), nitazoxanide is approximately 2 orders of magnitude less toxic than niclosamide (IC₅₀=5.2 μM). Similar to niclosamide, however, over-expression of the E. coli nitroreductases NfsA and NfsB (in this case overexpressed in 7KOΔtolC (DE3) from plasmid pET28; Novagen) is able to defend against nitazoxanide toxicity (FIG. 16).

### The Salicylamide Compound

Those skilled in the art will understand that any suitable salicylamide compound having antibiotic activity may be used in the combinations and compositions of the invention. Preferably the salicylamide compound exhibits antibiotic activity against Gram negative bacteria. Suitable salicylamide compounds for use in the present invention preferably include the structural moiety:

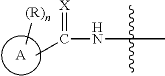

Where A is an aryl or heteroaryl ring, e.g. a phenyl ring, (R)ₙ indicates that the aryl or heteroaryl ring may optionally be substituted with one or more substituents, and X is oxygen or another heteroatom such as sulfur. The group —C(=x)-NH— can be linked to ring A via the carbon or the nitrogen atom. Preferably the salicylamide compound includes one or more nitro groups.

Preferably the salicylamide compound is a salicylanilide compound that includes two or more aryl groups, e.g. two or more phenyl rings, each of which may optionally be substituted, for example as shown in formula (I) below. Alternatively, the salicylamide compound may include one or more heteroaryl groups. The salicylamide compound may include a heteroatom, such as sulfur, in place of the oxygen of the amide group. The term “salicylamide compound” is intended to include all such analogues.

A preferred salicylamide compound is the salicylanilide compound niclosamide (N-(2′-chloro-4′-nitrophenyl)-5-chlorosalicylamide), the structure of which is shown below.

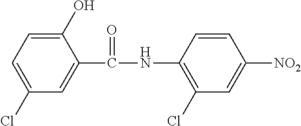 [SMILES: O=C(Nc1ccc([N+](=O)[O-])cc1Cl)c1cc(Cl)ccc1O]

Salt forms of niclosamide are known, including an ethanolamine salt and a piperizine salt. Furthermore, a monohydrate form of niclosamide is also known. Any suitable pharmaceutically acceptable salt or hydrate from may be used in the compositions and combinations of the present invention.

Other preferred salicylamide compounds include analogues of niclosamide. Such analogues are also known, for example those described in US2011/0183889, which is incorporated herein by reference. Suitable niclosamide analogues for use in the combinations and compositions of the present invention include, but are not limited to, those described by general formula (I), wherein R¹-R¹⁰ are as defined herein, including those listed in Table 1 below. Other suitable niclosamide analogues for use in the present invention include approved drug analogues of niclosamide.

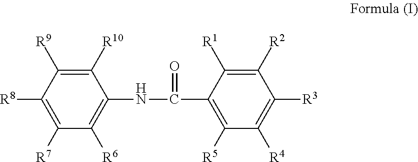

TABLE 1

| Compound | Substituents | | | | | | | | | |
|---|---|---|---|---|---|---|---|---|---|---|
| Number | R¹ | R² | R³ | R⁴ | R⁵ | R⁶ | R⁷ | R⁸ | R⁹ | R¹⁰ |
| 1 | OH | H | Cl | H | H | Cl | H | NO₂ | H | H |
| 2 | OH | Cl | H | H | H | Cl | H | NO₂ | H | H |
| 3 | OH | H | H | H | Cl | Cl | H | NO₂ | H | H |
| 4 | OH | H | H | Cl | H | H | Cl | NO₂ | H | H |
| 5 | OH | Cl | H | H | H | H | Cl | NO₂ | H | H |
| 6 | OH | H | Cl | H | H | H | Cl | NO₂ | H | H |
| 7 | OH | H | H | H | Cl | H | Cl | NO₂ | H | H |
| 8 | OH | H | H | Cl | H | Cl | NO₂ | H | H | H |
| 9 | OH | H | H | Cl | H | Cl | H | H | H | NO₂ |
| 10 | OH | H | H | H | Cl | Cl | H | H | NO₂ | H |
| 11 | H | OH | H | Cl | H | Cl | H | NO₂ | H | H |
| 12 | H | H | OH | Cl | H | Cl | H | NO₂ | H | H |
| 13 | Cl | OH | H | H | H | Cl | H | NO₂ | H | H |
| 14 | H | OH | Cl | H | H | Cl | H | NO₂ | H | H |
| 15 | H | OH | H | H | Cl | Cl | H | NO₂ | H | H |
| 16 | H | H | OH | H | Cl | Cl | H | NO₂ | H | H |
| 17 | OH | H | Cl | H | H | Cl | NO₂ | H | H | H |
| 18 | OH | Cl | H | H | H | Cl | NO₂ | H | H | H |
| 19 | OH | H | H | H | Cl | Cl | NO₂ | H | H | H |
| 20 | OH | H | H | Cl | H | F | H | NO₂ | H | H |
| 21 | OH | H | H | F | H | Cl | H | NO₂ | H | H |
| 22 | OH | H | H | Cl | H | Br | H | NO₂ | H | H |
| 23 | OH | H | H | Br | H | Cl | H | NO₂ | H | H |
| 24 | OH | H | H | Br | H | F | H | NO₂ | H | H |
| 25 | OH | H | H | F | H | Br | H | NO₂ | H | H |
| 26 | OH | H | H | Br | H | Br | H | NO₂ | H | H |
| 27 | OH | H | H | F | H | F | H | NO₂ | H | H |
| 28 | OH | H | H | Cl | H | Cl | H | NO₂ | H | H |
| 29 | OH | H | Cl | H | H | Br | H | H | H | H |
| 30 | H | H | OH | Cl | H | Br | H | NO₂ | H | H |
| 31 | OH | Cl | H | H | H | Br | H | NO₂ | H | H |
| 32 | OH | H | Cl | H | H | Cl | H | H | H | NO₂ |
| 33 | H | H | OH | Cl | H | Cl | H | H | H | NO₂ |
| 34 | OH | Cl | H | H | H | Cl | H | H | H | NO₂ |
| 35 | H | H | OH | F | H | Cl | H | H | H | NO₂ |
| 36 | H | H | OH | Br | H | Cl | H | H | H | NO₂ |
| 37 | H | OH | H | Cl | H | Cl | H | H | H | NO₂ |
| 38 | OH | H | Cl | H | H | F | H | NO₂ | H | H |
| 39 | H | H | OH | Cl | H | F | H | NO₂ | H | H |
| 40 | OH | Cl | H | H | H | F | H | NO₂ | H | H |
| 41 | H | OH | H | Cl | H | F | H | NO₂ | H | H |
| 42 | OH | H | H | H | Cl | Br | H | NO₂ | H | H |
| 43 | OH | H | H | H | Cl | F | H | NO₂ | H | H |
| 44 | OH | H | H | H | Cl | Cl | H | H | NO₂ | H |
| 45 | Cl | OH | H | H | H | Br | H | NO₂ | H | H |
| 46 | Cl | OH | H | H | H | Cl | NO₂ | H | H | H |
| 47 | Cl | OH | H | H | H | F | H | NO₂ | H | H |
| 48 | Cl | OH | H | H | H | H | Cl | NO₂ | H | H |
| 49 | Cl | OH | H | H | H | Cl | H | H | NO₂ | H |
| 50 | H | OH | Cl | H | H | Br | H | NO₂ | H | H |
| 51 | H | OH | Cl | H | H | Cl | NO₂ | H | H | H |
| 52 | H | OH | Cl | H | H | F | H | NO₂ | H | H |
| 53 | H | OH | Cl | H | H | H | Cl | NO₂ | H | H |
| 54 | H | OH | Cl | H | H | Cl | H | H | NO₂ | H |
| 55 | H | OH | H | H | Cl | Br | H | NO₂ | H | H |
| 56 | H | OH | H | H | Cl | Cl | NO₂ | H | H | H |
| 57 | H | OH | H | H | Cl | F | H | NO₂ | H | H |
| 58 | H | OH | H | H | Cl | H | Cl | NO₂ | H | H |
| 59 | H | OH | H | H | Cl | Cl | H | H | NO₂ | H |
| 60 | H | H | OH | H | Cl | Cl | NO₂ | H | H | H |
| 61 | H | H | OH | H | Cl | F | H | NO₂ | H | H |
| 62 | H | H | OH | H | Cl | H | Cl | NO₂ | H | H |
| 63 | H | H | OH | H | Cl | Cl | H | H | NO₂ | H |
| 64 | OH | H | Cl | H | H | Cl | H | H | NO₂ | H |
| 65 | H | H | OH | Cl | H | Cl | H | H | NO₂ | H |
| 66 | OH | Cl | H | H | H | Cl | H | H | NO₂ | H |
| 67 | OH | H | H | Br | H | Cl | H | H | NO₂ | H |
| 68 | OH | H | H | F | H | Cl | H | H | NO₂ | H |
| 69 | H | OH | H | Cl | H | Cl | H | H | NO₂ | H |

Other salicylamide compounds that are suitable for use in the combinations and compositions of the present invention include, but are not limited to, oxyclozanide (2,3,5-trichloro-N-(3,5-dichloro-2-hydroxyphenyl)-6-hydroxybenzamide), closantel (N-[5-Chloro-4-[(4-chlorophenyl)-cyanomethyl]-2-methylphenyl]-2-hydroxy-3,5-diiodobenzamide), rafoxanide (N-[3-chloro-4-(4-chlorophenoxy)phenyl]-2-hydroxy-3,5-diiodobenzamide), flusalan (3,5-dibromo-2-hydroxy-N-[3-(trifluoromethyl)phenyl]benzamide), tribromsalan (3,5-dibromo-N-(4-bromophenyl)-2-hydroxybenzamide), dibromsalan (5-Bromo-N-(4-bromophenyl)-2-hydroxybenzamide), resorantel (N-(4-bromophenyl)-2,6-dihydroxybenzamide), clioxanide (acetic acid 2-(4-chloro-phenylcarbamoyl)-4,6-diiodo-phenyl ester), 4′-chloro-5-nitrosalicylanilide, 2′-chloro-5′-methoxy-3-nitrosalicylanilide, 2′-methoxy-3,4′-dinitrosalicylanilide, 2′,4′-dimethyl-3-nitrosalicylanilide, 4′,5′-dibromo-3-nitrosalicylanilide, 2′-chloro-3,4′-dinitrosalicylanilide, 2′-ethyl-3-nitrosalicylanilide, 2′-bromo-3-nitrosalicylanilide.

The invention also includes the use of other salicylamide compounds, such as those containing one or more heteroaryl rings. The heteroaryl ring(s) may have one or more substituents. One example of such compounds is nitazoxanide (2-acetyloxy-N-(5-nitro 2-thiazolyl)benzamide), shown below.

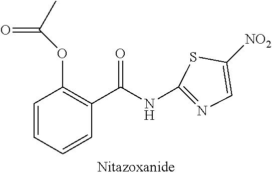 [SMILES: CC(=O)Oc1ccccc1C(=O)Nc1ncc([N+](=O)[O-])s1]

The invention furthermore includes the use of other salicylamide compounds, such as those where the oxygen of the amide group is replaced by another heteroatom. One example of such compounds is brotianide (3,4′-dibromo-5-chlorothiosalicylanilide) (shown below).

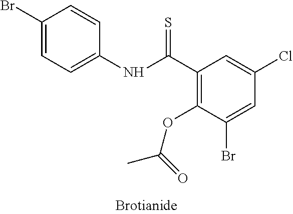 [SMILES: CC(=O)Oc1c(Br)cc(Cl)cc1C(=S)Nc1ccc(Br)cc1]

Some of the above-mentioned salicylamide compounds are commercially available. Others can readily be prepared by methods known to those skilled in the art. For example, WO 2004/006906, which is incorporated herein by reference, describes methods for preparing niclosamide analogues.

Those skilled in the art will understand that the salicylamide compound differs from the nitro-prodrug antibiotic, even though both classes of compound might include nitro group(s) within their chemical structures. The term “nitro-prodrug antibiotic” as used herein means a prodrug compound that is, initially upon administration, non-toxic or substantially non-toxic to bacteria, but undergoes reduction by one or more bacterial nitroreductase enzyme(s) thereby converting it to a drug that is toxic to bacteria. On the other hand, as surprisingly found by the applicants, a salicylamide compound of this invention, e.g. niclosamide, is one which is toxic to bacteria but is converted by one or more nitroreductase enzyme(s) to a species that is non-toxic to bacteria.

Preferably, a nitro-prodrug antibiotic compound that can be used in the present invention is a nitroimidazole derivative, although those skilled in the art will understand that other types of compounds may also be nitro-prodrug antibiotics. Examples of suitable nitro-prodrug antibiotics include, but are not limited to, nitrofurantoin, nitrofurazone, metronidazole, tinidazole, furazolidone, misonidazole, etanidazole, nifurtimox, ornidazole, benznidazole, dimetridazole, ronidazole, RSU-1069 (1-(1-aziridinyl)-3-(2-nitro-1-imidazolyl)-2-propanol), RB-6145 (1H-imidazole-1-ethanol, alpha-(((2-bromoethyl)amino)methyl)-2-nitro-, monohydrobromide), CB1954 (5-(aziridin-1-yl)-2,4-dinitrobenzamide), EF3 (2-(2-Nitroimidazol-1H-yl)-N-(3,3,3-trifluoropropyl)acetamide), EF5 (2-(2-nitro-1H-imidazol-1-yl)-N-(2,2,3,3,3-pentafluoropropyl)acetamide), HX4 (3-fluoro-2-(4-((2-nitro-1H-imidazol-1-yl)methyl)-1H-1,2,3-triazol-1-yl)-propan-1-ol) or fluorinated misonidazole.

### The Efflux Pump Inhibitor Compound

Efflux pumps are expressed in both Gram negative and Gram positive bacteria but are a more potent resistance mechanism in Gram negative bacteria. Homologues of the E. coli AcrAB-TolC efflux pump are thought to be the main efflux pumps of Gram negative bacteria. Efflux pump inhibitor compounds are those which interfere with the capability of an efflux pump to export another compound, e.g. an antibiotic, from a cell. It is known that delivering an efflux pump inhibitor compound together with an antibiotic can increase the potency of the antibiotic, even against strains that have been identified as resistant. Such efflux inhibitor compounds may be competitive inhibitors of the efflux pump. For example, PAβN (phenylalanine-arginine β-napthylamide) is a competitive inhibitor of AcrAB-TolC, meaning it is preferentially exported out of the cell, thereby reducing the rate of antibiotic export and allowing the antibiotic to accumulate to a level that is toxic.

Those skilled in the art will realise that any suitable efflux pump inhibitor compound may be used in the combinations and compositions of the present invention. Preferred efflux pump inhibitor compounds are those which are active over a broad range of bacterial strains, particularly those which are active against efflux pumps of Gram negative bacteria such as homologues of the E. coli AcrAB-TolC efflux pump. WO 96/33285, which is incorporated herein by reference, describes methods for screening for inhibitors of microbial efflux pump inhibitors. Those skilled in the art will recognise that such screening methods can be used to identify efflux pump inhibitors that may be employed in the present invention.

Suitable efflux pump inhibitor compounds for use in the combinations and compositions of the present invention include, but are not limited to, those described in U.S. Pat. No. 6,399,629, which is incorporated herein by reference. Such efflux pump inhibitor compounds include, but are not limited to, (2R,4S)-4-(2-aminoacetamido)-N-[(1R-)-3-phenyl-1-(3-quinolylcarbamoyl)propyl]-2-pyrrolidinecarboxamide, (2R,4S)-4-(2-aminoacetamido)-N-[(1R)-3-phenyl-1-(3-phenyl)propylcarbamoyl]propyl]-2-pyrrolidinecarboxamide, (2S,4R)-4-(2-aminoacetamido)-N-[(1R)-3-phenyl-1-(3-quinolylcarbamoyl)propyl]-2-pyrrolidinecarboxamide, (2S,4R)-4-(2-aminopropionamido]-N-[(1R)-3-phenyl-1-(3-quinolylcarbamoyl)propyl]-2-pyrrolidinecarboxamide, (2S,4R)-4-(aminompropionamido)-N-[(1R)-3-phenyl-1-(3-quinolylcarbamolyl)propyl]-2-pyrrolidinecarboxamide, (2S,4R)-4-[3-(aminopropionamido])-N-[(1R)-3-phenyl-1-(3-quinolylcarbamoyl)propyl]-2-pyrrolidinecarboxamide, (2S,4R)-4-(amino-N-[(1R)-3-pheynl-1-(3-quinolylcarbamoyl)propyl]-2-pyrrolidinecarboxamide, (2S,4R)-4-(aminoacetamido)-N-[(1R)-3-methyl-1-[(3-quinolylcarbamoyl)butyl]-2-pyrrolidinecarboxamide, (2S,4S-4-(2-amino-N-methylacetamido)-N-[(1R)-3-phenyl-1-(3-quinolylcarbamoyl)propyl]-2-pyrrolidinecarboxamide, (2S,4R)-4-(2-aminoacetamido)-N-[(1R)-3-3-methyl-1-(6-methoxy-8-methyl-3-quinolylcarbamoyl)butyl]-2-pyrrolidinecarboxamide, (2S,4R)-4-(2-aminoacetamido)-N-[(1R)-3-phenyl-1-(6,7-dimethyl-3-quinolylcarbamoyl)propyl]-2-pyrrolidinecarboxamide, (2S,4R)-4-(2-aminoacetamido)-N-[(1R)-2-(4-methoxyphenyl)-1-(3-quinolylcarbamoyl)ethyl]-2-pyrrolidinecarboxamide, (2R,4R)-4-(aminomethyl)-N-[(1R)-3-methyl-1-quinolylcarbamoyl)bulyl]-2-pyrrolidinecarboxamide, (2R,4R)-4-aminomethyl)-N-[(1R)-3-phenyl-1-(6-ethyl-3-quinolylcarbamoyl)propyl]-2-pyrrolidinecarboxamide, (2S,4R)-4-(2-aminoacetamido)-N-methyl-N-[(1R)-3-phenyl-1-(3-quinolylcarbamoyl)propyl]-2-pyrrolidinecarboxamide, (2R,4R)-4-(aminomethyl)-N-[(1R)-3-phenyl-1-(3-quinolylcarbamoyl)propyl]-2-pyrrolidinecarboxamide, (2R,4R)-4-(2-aminoacetamido)-N-[(1R)-3-phenyl-1-(3-quinolylcarbamoyl)propyl]-2-pyrrolidinecarboxamide, (2S,4R)-4-(2-aminoacetamido)-N-[(1R)-3-phenyl-1-(6-ethyl-3-quinolylcarbamoyl)propyl]-2-pyrrolidinecarboxamide, (2S,4R)-4-(2-aminoacetamido)-N-[(1R)-3-methyl-1-(6-ethyl-3-quinolylcarbamoyl)butyl]-2-pyrrolidinecarboxamide, (2R,4R)-4-(2-aminoacetamido)-N-[(1R)-2-(4-fluorophenyl)-1-(3-quinolylcarbamoyl)ethyl]-2-pyrrolidinecarboxamide, (2S,4R)-4-(2-aminoacetamido)-N-[(1R)-2-(4-fluorophenyl)-1-(3-quinolylcarbamoyl)ethyl]-2-pyrrolidinecarboxamide, (2S,4R)-1-(2-aminoacetamido)-N-[(1R)-3-phenyl-1-(6-methoxy-8-methyl-3-quinolylcarbamoyl)propyl]2-pyrrolidinecarboxamide, (2S,4R)-4-(2-aminoacetamido)-N-[(1R)-2-(4-hydroxyphenyl)-1-(3-quinolylcarbamoyl)ethyl]-2-pyrrolidinecarboxamide, (2S,4S)-4-(aminomethyl)-N-[(1R)-3-phenyl-1-(3-quinolylcarbamoyl)propyl]-2-pyrrolidinecarboxamide, (2R,4R)-4-(aminomethyl)-N-[(1R)-2-(4-hydroxyphenyl)-1-(6-ethyl-3-quinolylcarbamoyl)ethyl]-2-pyrrolidinecarboxamide, (2S,4R)-4-(2-aminoacetamido)-N-[(1R)-3-phenyl-1-(5-chloro-2-hydroxyphenylcarbamoyl)propyl]-2-pyrrolidinecarboxamide, (2S,4R)-4-(2-aminoacetamido)-N—R1R)-3-phenyl-1-(4,5-dimethyl-2-hydroxyphenylcarbamoyl)propyl]-2-pyrrolidinecarboxamide, (2S,4R)-4-(2-aminoacetamido)-N-[(1R)-3-phenyl-1-(2-hydroxy-5-methylphenylcarbamoyl)propyl]-2-pyrrolidinecarboxamide, (2R,4R)-4-(aminomethyl)-N-[(1R)-3-phenyl-1-(6-quinolylcarbamoyl)propyl]-2-pyrrolidinecarboxamide, (2R,4R)-4-(aminomethyl)-N-[(1R)-3-phenyl-1-(8-quinolylcarbamoyl)propyl]-2-pyrrolidinecarboxamide, (2R,4R)-4-(aminomethyl)-N-[(1R)-2-(4-fluorophenyl)-1-(3-quinolylcarbamoyl)ethyl]-2-pyrrolidinecarboxamide, (2R,4R)-4-(aminomethyl)-N-[(1R)-3-phenyl-[3-quinolylmethyl)carbamoyl]propyl]-2-pyrrolidinecarboxamide, (2R,4R)-4-(aminomethyl)-N-methyl-N-[(1R)-3-phenyl-1-(3-quinolylcarbamoyl)propyl]-2-pyrrolidinecarboxamide, (2R,4R)-4-(aminomethyl)-N-[(1R)-3-methyl-1-(3-quinolylcarbamoyl)butyl]-2-pyrrolidinecarboxamide, (2R,4R)-4-(aminomethyl)-N-[(1R)-3-phenyl-1-(6-fluoro-3-quinolylcarbamoyl)propyl]-2-pyrrolidinecarboxamide, (2R,4R)-4-(aminomethyl)-N-[(1R)-4-methyl-1-(3-quinolylcarbamoyl)pentyl]-2-pyrrolidinecarboxamide, (2R,4R)-4-(aminomethyl)-N-[(1R)-3-phenyl-1-(5-fluoro-2-hydroxyphenylcarbamoyl)propyl]-2-pyrrolidinecarboxamide, (2R,4R)-4-(aminomethyl)-N-[(1R)-3-phenyl-1-(2-hydroxy-5-methylphenylcarbamoyl)propyl]-2-pyrrolidinecarboxamide, (2R,4R)-4-(aminomethyl)-N-[(1R)-3-phenyl-1-(5-chloro-2-hydroxyphenylcarbamoyl)propyl]-2-pyrrolidinecarboxamide, (2R,4R)-4-(aminomethyl)-N-methyl-N-[(1R)-3-methyl-1-(6-ethyl-3-quinolylcarbamoyl)butyl]-2-pyrrolidinecarboxamide, (2R,4S-4-(2-aminoacetamido)-N-(2-phenylethyl)-N-(3-quinolylcarbamoyl)ethyl]-2-pyrrolidinecarboxamide, (2R,4S)-4-[(2R)-2-aminopropionamido]-N-(2-phenylethyl)-N-(3-quinolylcarbamoyl)ethyl]-2-pyrrolidinecarboxamide, (2S,4R)-4-(2-aminoacetamido)-N-(2-phenylethyl)-N-(3-quinolylcarbamoyl)ethyl]-2-pyrrolidinecarboxamide, (2R,4S)-4-(2-aminoacetamido)-N-(2-methylpropyl)-N-(7-ethyl-3-quinolylcarbamoyl)ethyl]-2-pyrrolidinecarboxamide, (2R,4R)-4-(aminomethyl)-N-(2-phenylethyl)-N-(3-quinolylcarbamoyl)ethyl]-2-pyrrolidinecarboxamide, (2R,4R)-4-(2-aminoacetamido)-N-(2-phenylethyl)-N-(3-quinolylcarbamoyl)methyl]-2-pyrrolidinecarboxamide, (2R,4S)-4-[(2R)-2-aminopropionamido]-N-(3,3-dimethylbutyl)-N-(3-quinolylcarbamoyl)methyl]-2-pyrrolidinecarboxamide, (2R,4R)-4-(aminomethyl)-N-[(1R)-3-phenyl-1-[(2-quinolyloxy)methyl]propyl]-2-pyrrolidinecarboxamide, (2R,4S)-4-(2-aminoacetamido)-N-[(1R)-3-methyl-1-[(2-naphthyloxy)methyl]butyl]-2-pyrrolidinecarboxamide, (2S,4R)-4-(2-aminoacetamido)-N-[(1R)-3-phenyl-1-[(4-chlorophenylthio)methyl]propyl]-2-pyrrolidinecarboxamide, (2S,4R)-4-[(2R)-2-aminopropionamido]-N-[(1R)-3-phenyl-1-[(4-chlorophenylthio)methyl]propyl]-2-pyrrolidinecarboxamide, (2R,4S)-4-[(2R)-2-aminopropionamido]-N-[(1R)-3-phenyl-1-[(4-chlorophenylthio)methyl]propyl]-2-pyrrolidinecarboxamide, (2S,4R)-4-(2-aminoacetamido)-N-[(1R)-3-phenyl-1-[(3-quinolylthio)methyl]propyl]-2-pyrrolidinecarboxamide, (2S,4R)-4-(2-aminoacetamido)-N-[(1R)-3-phenyl-1-[(2-quinolyloxy)methyl]propyl]-2-pyrrolidinecarboxamide, (2S,4R)-4-(2-aminoacetamido)-N-[(1R)-3-phenyl-1-[(2-quinolylthio)methyl]propyl]-2-pyrrolidinecarboxamide, (2S,4R)-4-(2-aminoacetamido)-N-[(1R)-3-phenyl-1-(phenylthiomethyl)propyl]-2-pyrrolidinecarboxamide, (2S,4R)-4-(2-aminoacetamido)-N-[(1R)-3-phenyl-1-[(4-fluorophenylthio)methyl]propyl]-2-pyrrolidinecarboxamide, (2S,4R)-4-(2-aminoacetamido)-N-[(1R)-3-methyl-1-[(2-quinolyloxy)methyl]butyl]-2-pyrrolidinecarboxamide, (2R,4R)-4-(aminomethyl)-N-[(1R)-3-methyl-1-[(3-quinolyloxy)methyl]butyl]-2-pyrrolidinecarboxamide, (2S,4R)-4-(2-aminoacetamido)-N-[(1R)-3-methyl-1-[(4-chlorophenylthio)methyl]butyl]-2-pyrrolidinecarboxamide, (2R,4R)-4-(aminomethyl)-N-[(1R)-3-methyl-1-[(4-chlorophenylthio)methyl]butyl]-2-pyrrolidinecarboxamide, (2R,4R)-4-(aminomethyl)-N-[(2S)-2-(6-methyl-3-quinolylcarboxamido)-4-phenylbutyl]-2-pyrrolidinecarboxamide, (2R,4R)-4-(aminomethyl)-N-[1R)-3-phenyl-1-[(6-methyl-3-quinolylcarboxamido)methyl]propyl]-2-pyrrolidinecarboxamide, (2S,4R)-4-(2-aminoacetamido)-N-[1R)-1-[(RS)-(5,6-dimethyl-2-benzoxazolyl)hydroxymethyl]-3-phenylpropyl]-2-pyrrolidinecarboxamide, (2S,4R)-4-[(2R)-2-aminopropionamido]-N-[(1R)-1-[(RS)-(5,6-dimethyl-2-benzoxazolyl)hydroxymethyl]-3-phenylpropyl]-2-pyrrolidinecarboxamide, (2R,4S)-4-[(2R)-2-aminopropionamido]-N-[(1R)-1-[(RS)-(5,6-dimethyl-2-benzoxazolyl)hydroxymethyl]-3-phenylpropyl]-2-pyrrolidinecarboxamide, (2S,4R)-4-(2-aminoacetamido)-N-[(1R)-1-[5,6-dimethyl-2-benzoxazolyl)hydroxymethyl]-3-phenylpropyl]-2-pyrrolidinecarboxamide, (2R,4R)-4-(aminomethyl)-N-[(1R)-1-[(RS)-(5-1,1-dimethyl)ethyl-2-benzoxazolyl)hydroxymethyl]-3-phenylpropyl]-2-pyrrolidinecarboxamide, (2R,4R)-4-(aminomethyl)-2-[((1S)-3-phenyl-1-(3-quinolylcarbamoyl)propyl)oxymethyl]pyrrolidine, (2R,4R)-4-(aminomethyl)-2-[((1R)-3-phenyl-1-(3-quinolylcarbamoyl)propyl)oxymethyl]pyrrolidine, (2R,4R)-4-(aminomethyl)-2-(2-quinolyloxymethyl)pyrrolidine, (2R,4R)-4-(aminomethyl)-2-(6-methyl-3-quinolylcarboxamidomethyl)pyrrolidine, (2S,4R)-4-(2-aminoacetamido)-2-(5-benzyl-2-benzimidazolyl)pyrrolidine, (2R,4R)-4-(aminomethyl)-2-(5-benzyl-2-benzimidazolyl)pyrrolidine, (2S,4R)-4-(2-aminoacetamido)-2-(1-(2-phenyl)ethyl-2-benzimidazolyl)pyrrolidine, (2S,4R)-4-(2-aminoacetamido)-2-(1-(3-aminopropyl)-2-benzimidazolyl)pyrrolidine, (2S,4R)-4-(2-aminoacetamido)-N-[(1R)-1-(2-benzoxazolyl)-3-phenylpropyl]-2-pyrrolidinecarboxamide, (2S,4R)-4-(2-aminoacetamido-N-[(1S)-1-(2-benzimidazolyl)-3-phenylpropyl]-2-pyrrolidinecarboxamide, (2S,4R)-4-(2-aminoacetamido)-N-[(5-benzyl-2-benzimidazolyl)methyl]-2-pyrrolidinecarboxamide, (2S,4R)-4-(2-aminoacetamido)-N-[(1-(2-phenyl)ethyl-2-benzimidazolyl)methyl]-2-pyrrolidinexarboxamide, (2S,4R)-4-(2-aminoacetamido)-N-[(5-1,1-dimethyl)ethyl-2-benzimidazolyl)methyl]-2-pyrrolidinecarboxamide, (2S,4R)-4-(2-aminoacetamido)-N-[(5-(1-hydroxy-1-phenyl)methyl-2-benzimidazolyl)methyl]-2-pyrrolidinecarboxamide, (2S,4R)-4-(2-aminoacetamido)-N-[(1S)-1-(5-benzyl-2-benzimidazolyl)ethyl]-2-pyrrolidinecarboxamide, (2S,4R)-4-(2-aminoacetamido)-N-[(1R)-1-(2-benzthiazolyl)-3-phenylpropyl]-2-pyrrolidinecarboxamide. (2S,4R)-4-(2-aminoacetamido)-N-[(1S)-1-(2-benzoxazolyl)-3-phenylpropyl]-2-pyrrolidinecarboxamide, (2R,4R)-4-(aminomethyl)-N-[(5-benzyl-2-benzimidazolyl)methyl]-2-pyrrolidinecarboxamide, (2R,4S)-4-(aminomethyl)-N-[(5-benzyl-2-benzimidazolyl)methyl]-2-pyrrolidinecarboxamide, (2R,4R)-4-(aminomethyl)-N-[(5-phenyloxy-2-benzimidazolyl)methyl]-2-pyrrolidinecarboxamide, (2R,4R)-4-(aminomethyl)-N-[(5-phenyl-2-benzimidaxolyl)methyl]-2-pyrrolidinecarboxamide, (2S,4R)-4-(2-aminoethylthio)-N-[(1R)-3-phenyl-1-(3-quinolylcarbamoyl)propyl]-2-pyrrolidinecarboxamide, (2S,4R)-4-(2-aminoethyloxy)-N-[(1R)-3-phenyl-1-(3-quinolylcarbamoyl)propyl]-2-pyrrolidinecarboxamide, (2S,4R)-4-(2-aminoethyloxy)-N-(6-(1,1-dimethyl)ethyl-3-quinolyl)-2-pyrrolidinecarboxamide, (2S,4RS)-4-(3-aminopropyl)-N-[(1R)-3-phenyl-1-(3-quinolylcarbamoyl)propyl]-2-pyrrolidinecarboxamide, (2S,4R)-4-(2-aminoacetamido)-N-[5-(p-chlorophenyl)tetrahydro-3-thienyl]-2-pyrrolidinecarboxamide, (2S,4R)-4-(guanidinyl)-N-[(1R)-3-phenyl-1-(3-quinolylcarbamoyl)propyl]-2-pyrrolidinecarboxamide, (2S,4R)-4-(2-aminoacetamido)-N-[7-ethyl-3-quinolyl]-2-pyrrolidinecarboxamide, (2R,4R)-4-(aminomethyl)-N-[6-(1,1-dimethyl)ethyl-3-quinolyl]-2-pyrrolidinecarboxamide, (2S,4R)-4-(2-aminoacetamido)-N-(5-benzyl-2-hydroxyphenyl)-2-pyrrolidinecarboxamide, (2S,4R)-4-(2-aminoacetamido)-N-[4-benzyl-2-benzimidazolyl)ethyl]-2-pyrrolidinecarboxamide, (2R,4R)-4-(aminomethyl)-N-(6-ethyl-3-quinolyl)-2-pyrrolidinecarboxamide, (2R,4R)-4-(aminomethyl)-N-(5-benzyl-2-benzimidazolyl)-2-pyrrolidinecarboxamide, (2S,4R)-4-(2-aminoacetamido)-N-(5-benzyl-2-benzimidazolyl)-2-pyrrolidinecarboxamide, (2R,4R)-4-(aminomethyl)-N-[(1R)-3-phenyl-1-[(3-quinolylcarboxamido)methyl]propyl]-2-pyrrolidinecarboxamide, trans-4-glycylamino-D-prolyl-D-proline-(6-isopropyl)-3-quinolylamide, trans-4-amino-L-prolyl-trans-4-((R)-2-hydroxy-4-phenylbutyrylamino-L-proline 3-quinolylamide, trans-4-glycylamino-L-prolyl-trans-4-((R)-2-hydroxy-4-phenylbutrylamino)-L-proline 3-quinolylamide, cis-4-glycylamino-L-prolyl-trans-4-((R)-2-hydroxy-4-phenylbutyrylamino)-L-proline 3-quinolylamide, trans-4-glycylamino-D-prolyl-trans-4-((R)-2-hydroxy-4-phenylbutyrylamino)-L-proline 3-quinolylamide, trans-4-(N-methylglycylamino)-L-prolyl-trans-4-((R)-2-hydroxy-4-phenylbutyrylamino)-L-proline 3-quinolylamide, trans-4-((S)-3-amino-2-hydroxypropionylamino)-L-prolyl-trans-4-((R)-2-hydroxy-4-phenylbutyrylamino)-L-proline 3-quinolylamide, trans-4-aminomethyl-L-prolyl-trans-4-((R)-2-hydroxy-4-phenylbutyrylamino)-L-proline 3-quinolylamide, 4-(2-aminoethyl)-L-prolyl-trans-4-((R)-2-hydroxy-4-phenylbutyrylamino)-L-proline 3-quinolylamide, 1-(N-methylglycyl)-trans-4-amino-L-prolyl-trans-4-((R)-2-hydroxy-4-phenylbutyrylamino)-L-proline 3-quinolylamide, trans-4-amino-L-pipecolinoyl-trans-4-((R)-2-hydroxy-4-phenylbutyrylamino)-L-proline 3-quinolylamide, cis-4-amino-L-pipecolinoyl-trans-4-((R)-2-hydroxy-4-phenylbutyrylannino)-L-proline 3-quinolylamide, trans-4-glycylamino-L-pipecolinoyl-trans-4-((R)-2-hydroxy-4-phenylbutyrylamino)-L-proline 3-quinolylamide, cis-4-glycylamino-L-pipecolinoyol-trans-4-((R)-2-hydroxy-4-phenylbutyrylamino)-L-proline 3-quinolylamide, D-ornithyl-trans-4-(4-phenylbutanoyl)amino-L-proline-5-indanylamide, L-ornithyl-cis-4-(4-phenylbutanoyl)amino-L-proline-5-indanylamide, D-ornithyl-cis-4-(4-phenylbutanoyl)amino-L-proline 5-indanylamide, 4-hydroxy-L-ornithyl-trans-4-(4-phenylbutanoyl)amino-L-proline 5-indanylamide, trans-4-glycylamino-L-prolyl-D-homophenylalanine 3-quinolylamide, trans-4-amino-L-prolyl-D-homophenylalanine 3-quinolylamide, trans-4-glycylamino-L-prolyl-D-homophenylalanine 5-indanylamide, trans-4-glycylamino-L-prolyl-D-homophenylalanine 3,4-dimethylphenylamide, trans-4-glycylamino-L-prolyl-D-homophenylalanine 3,5-dimethylphenylamide, trans-4-glycylamino-L-prolyl-D-homophenylalanine 4-chloro-3-methylphenylamide, trans-4-glycylamino-L-prolylglycine 4-benzylphenylamide, trans-4-glyclamino-L-proline 4-phenoxyphenylamide, trans-4-glycylamino-L-proline 4-(4′-methylphenoxy)phenylamide, trans-4-glycylamino-L-proline 4-(4′-chlorophenoxy)phenylamide, trans-4-glycylamino-L-proline 4-phenylaminophenylamide, trans-4-glycylamino-L-proline 3-biphenylamide, trans-4-glycylamino-D-proline 3-biphenylamide, trans-4-glycylamino-L-proline 4-benzylphenylamide, trans-4-glycylamino-L-proline 4-tert-butylphenylamide, trans-4-glycylamino-L-proline 4-phenylbenzylamide, trans-4-glycylamino-L-proline 4-benzyloxyphenylamide, trans-4-glycylamino-L-proline 3-benzyloxyphenylamide, trans-4-glycylamino-L-proline 4-(phenylthiomethyl)phenylamide, trans-4-glycylamino-L-proline 4-benzylthiophenylamide, trans-4-((S)-3-amino-2-hydoxypropionylamino)-L-proline 4-phenoxyphenylamide, trans-4-(2-aminoethylsulfonylamino)-L-proline 4-phenoxyphenylamide, trans-4-glycylamino-L-proline 4-phenylthiazol-2-ylamide, trans-4-glycylamino-L-proline 3-(6-benzyl)quinolylamide, trans-4-amino-L-pipecolinoyl-(4-phenoxyphenyl)amide, trans-4-glycylamino-L-pipecolinoyl 4-phenoxyphenylamide, trans-4-aminomethyl-L-proline 4-phenoxyphenylamide, 1-(trans-4-glycylamino-L-prolyl)-4-(3-chlorophenyl)piperazine, 1-[trans-4-((2S)-3-amino-2-hydroxypropionylamino)-D-prolyl]-4-(3-chloro-2-methylphenyl)piperazine, 1-(N-trans-4-glycylamino-L-prolyl)-4-(4-chlorophenyl)piperazine, 1-(trans-4-glycylamino-L-prolyl)-4-(2-chlorophenyl)piperazine, 1-(trans-4-aminomethyl-L-prolyl)-4-(3-chloro-2-methylphenyl)piperazine, 1-(trans-4-glycylamino-L-prolyl)-4-(4-phenylbutanoyl)piperazine, (2R)-4-benzyl-1-(trans-4-glycylamino-D-prolyl)-2-phenethylpiperazine, 1-(trans-4-glycylamino-L-prolyl)-4-(4-benzyloxyphenoxy)piperidine, 1-(trans-4-glycylamino-L-prolyl)-4-(3,5-dichlorophenoxy)piperidine, 1-(trans-4-glycylamino-D-prolyl)-4-(3,5-dichlorophenoxy)piperidine, trans-4-glycylamino-L-prolyl-4-(2-chloro-5-methylphenoxy)piperidine, (2S,4R)-4-glycylamino-2-(4-biphenyloxy)methylpyrrolidine, (2S,4R)-4-glycylamino-2-(3-biphenyloxy)methylpyrrolidine, (2R,4S)-4-glycylamino-2-(4-biphenyloxy)methylpyrrolidine, (2R,4S)-4-glycylamino-2-(3-biphenyloxy)methylpyrrolidine, trans-4-(3-biphenyloxy)-L-proline 2-aminoethylamide, (2S,4R)-2-(2-amino-1-hydroxyethyl)-4-(3-biphenyloxy)pyrrolidine, 1-(N-trans-4-glycylamino-L-prolylamino)-3-(4-phenylpropanoylamino)benzene, 2-(trans-4-glycylamino-L-prolylamino)-6-(4-phenylpropanoylamino)pyridine or (2S,4R)-4-glycylamino-2-((E and Z)-4-phenylstyryl)pyrrolidine.

Other efflux pump inhibitor compounds that are suitable for use in the combinations and compositions of the present invention include, but are not limited to, globomycin (glycine, N—(N—(N—(N—(N-(3-hydroxy-2-methyl-1-oxononyl)-N-methylleucyl)-L-alloisoleucyl)-L-seryl)-L-allothreonyl)-, rho-lactone), carbonyl cyanide m-chlorophenylhydrazone (CCCP), pyridoquinolone, MC-04,124 ((2R,4R)-4-(aminomethyl)-N-[(2R)-1-oxo-4-phenyl-1-(quinolin-6-ylamino)butan-2-yl]pyrrolidine-2-carboxamide), or MC-02,595 (D-ornithine-D-homophenylalanine-3-aminoquinoline).

Classes of efflux pump compounds that are suitable for use in the combinations and compositions of the present invention include, but are not limited to, alkoxyquinoline derivatives, e.g. 2,8-dimethyl-4-(2′-pyrrolidinoethyl)-oxyquinoline; piperidine and piperidine analogues; phenothiazines, e.g. chloropromazine; monoterpene derivatives, e.g. geranylamine; or arginine derivatives such as those described in U.S. Pat. No. 6,251,869, which is

Another example of an efflux pump inhibitor that is suitable for use in the combinations and compositions of the present invention is 2-3 dibromomaleimide.

A preferred efflux pump inhibitor that is suitable for use in the combinations and compositions of the present invention is phenylalanine-arginine β-napthylamide (PAβN).

### Other Aspects

The salicylamide compound and the efflux pump inhibitor compound may be administered separately, sequentially or simultaneously. For example, the combination of the salicylamide compound and the efflux pump inhibitor compound may be formulated together as a composition for administration to a patient. Alternatively, the salicylamide compound and the efflux pump inhibitor compound may each be separately formulated for separate or sequential administration to a patient.

The salicylamide compound or the salicylamide compound and the efflux pump inhibitor compound may be administered to a patient by a variety of routes, including orally, parenterally, by inhalation spray, topically, rectally, nasally, buccally, intravenously, intra-muscularly, intra-dermally, subcutaneously or via an implanted reservoir, preferably intravenously. The amount of each compound to be administered will vary widely according to the nature of the patient and the nature and extent of the disorder to be treated. Typical dosages for an adult human will be up to about 5 g, preferably up to about 2 g, for the salicylamide compound and up to about 5 g, preferably up to about 2 g, for the efflux pump inhibitor compound. The specific dosages required for any particular patient will depend upon a variety of factors, including the patient's age, body weight, general health, sex, etc.

For separate, sequential or simultaneous oral administration the salicylamide compound and the efflux pump inhibitor compound can be formulated into solid or liquid preparations, for example tablets, capsules, powders, solutions, suspensions and dispersions. Such preparations are well known in the art as are other oral dosage regimes not listed here. In the tablet form the compounds may be tableted with conventional tablet bases such as lactose, sucrose and corn starch, together with a binder, a disintegration agent and a lubricant. The binder may be, for example, corn starch or gelatin, the disintegrating agent may be potato starch or alginic acid, and the lubricant may be magnesium stearate. For oral administration in the form of capsules, diluents such as lactose and dried corn-starch may be employed. Other components such as colourings, sweeteners or flavourings may be added.

When aqueous suspensions are required for oral use, the salicylamide compound or the salicylamide compound and the efflux pump inhibitor compound may be combined with carriers such as water and ethanol, and emulsifying agents, suspending agents and/or surfactants may be used. Colourings, sweeteners or flavourings may also be added.

The salicylamide compound or salicylamide compound and the efflux pump inhibitor compound may also be administered separately, sequentially or simultaneously, by injection in a physiologically acceptable diluent such as water or saline. The diluent may comprise one or more other ingredients such as ethanol, propylene glycol, an oil or a pharmaceutically acceptable surfactant. In one example, the compounds are administered separately, sequentially or simultaneously by intravenous injection, where the diluent comprises an aqueous solution of sucrose, L-histidine and a pharmaceutically acceptable surfactant, e.g. Tween 20.

The salicylamide compound or salicylamide compound and the efflux pump inhibitor compound may also be administered, separately, sequentially or simultaneously, topically. Carriers for topical administration of the compounds include mineral oil, liquid petrolatum, white petrolatum, propylene glycol, polyoxyethylene, polyoxypropylene compound, emulsifying wax and water. The compounds may be present as ingredients in lotions or creams, for topical administration to skin or mucous membranes. Such creams may contain the active compounds suspended or dissolved in one or more pharmaceutically acceptable carriers. Suitable carriers include mineral oil, sorbitan monostearate, polysorbate 60, cetyl ester wax, cetearyl alcohol, 2-octyldodecanol, benzyl alcohol and water.

The salicylamide compound or salicylamide compound and the efflux pump inhibitor compound may further be administered separately, sequentially or simultaneously, by means of sustained release systems. For example, they may be incorporated into slowly dissolving tablets or capsules.

For the treatment of infections in plants, for example bacterial infections caused by Pseudomonas syringae pv. actinidiae (Psa-V) in kiwifruit plants of the genus Actinidia, the salicylamide compound and the efflux pump inhibitor compound may optionally be formulated with one or more carriers, for example as a spray for application to plants. The compounds may be applied separately, sequentially or simultaneously. For application to plants, the combinations and compositions of the invention may further comprise one or more adjuvants, such as emulsifiers, dispersants, mineral and vegetable oils, or mixtures thereof suitable for application to plants. The combinations and compositions can also be used as sterilising agents for field equipment (e.g. pruning shears), to prevent spreading of bacterial infections between orchards.

The present invention also relates to devices and kits for treating or preventing bacterial infections. Suitable kits comprise at least one salicylamide compound and at least one efflux pump inhibitor compound sufficient for at least one treatment of at least one bacterial infection, for separate, sequential or simultaneous use, together with instructions for performing the treatment/prevention.

The instructions for use of the kit and treating/preventing the bacterial infection can be in the form of labelling, which refers to any written or recorded material that is attached to, or otherwise accompanies a kit at any time during its manufacture, transport, sale or use. For example, the term “labelling” encompasses advertising leaflets and brochures, packaging materials, instructions, audio or video cassettes, computer discs, as well as writing imprinted directly on kits.

The applicants' results presented herein provide surprisingly interesting insight into the molecular basis of how bacteria metabolise certain drugs, including nitro-prodrug antibiotics as well as salicylamides that contain one or more nitro groups, e.g. niclosamide and niclosamide analogs. Without wishing to be bound by theory, the applicants propose that bacteria that have become resistant to treatment with nitro-prodrug antibiotics would then be susceptible to treatment with one or more nitro group-containing salicylamide compounds owing to spontaneous mutation in endogenous nitroreductase genes. On the one hand, loss of, or reduction in, endogenous nitroreductase activity compared to wild type means that the bacterial cell is resistant to nitro-prodrug antibiotics, because once inside the bacterial cell the prodrug has no way of being cleaved to produce the toxic antibiotic. On the other hand, loss of, or reduction in, endogenous nitroreductase activity means that the bacterial cell is more susceptible to one or more nitro group-containing salicylamide compounds, for example niclosamide and niclosamide analogs, because in the absence of nitroreductase activity the bacterial cell is no longer capable of converting the toxic niclosamide to a non-toxic form. An effux pump inhibitor may optionally be included with the one or more nitro group-containing salicylamide compounds to enhance sensitivity to the drug.

Accordingly, in yet another aspect the present invention provides a method for treating or preventing a bacterial infection in a patient, wherein the bacteria have become resistant to treatment with a nitro-prodrug antibiotic, comprising administering to the patient at least one salicylamide compound in an amount sufficient to treat or prevent infection, wherein the salicylamide compound includes one or more nitro group. Optionally, the method further comprises administering at least one efflux pump inhibitor.

In yet another aspect the present invention provides a method for reducing or eliminating formation of a bacterial biofilm, wherein the bacteria have become resistant to treatment with a nitro-prodrug antibiotic, comprising administering at least one salicylamide compound in an amount sufficient to reduce or eliminate formation of the biofilm, salicylamide compound includes one or more nitro group. Optionally, the method further comprises administering at least one efflux pump inhibitor.

Conversely, bacteria that have become resistant to treatment with one or more nitro group-containing salicylamide compounds, with or without an efflux pump inhibitor present, may have done so via mutations in endogenous nitroreductase genes that cause an increase in nitroreductase enzyme activity. On the one hand, an increase in endogenous nitroreductase activity compared to wild type means that the bacterial cell is resistant to nitro group-containing salicylamide compounds (in the presence of absence of an efflux pump inhibitor) because the bacterial cell is no longer capable of converting the toxic nitro group-containing salicylamide compound, for example niclosamide and niclosamide analogs, to a non-toxic form. On the other hand, an increase in endogenous nitroreductase activity means that the bacterial cell is more susceptible to one or more nitro-prodrug antibiotics, because it will cleave the prodrug to form an active form of the antibiotic.

Accordingly, in yet another aspect the present invention provides a method for treating or preventing a bacterial infection in a patient, wherein the bacteria have become resistant to treatment with at least one salicylamide compound and at least one efflux inhibitor compound, wherein the salicylamide compound includes one or more nitro groups, comprising administering to the patient a nitro-prodrug antibiotic in an amount sufficient to treat or prevent the infection.

In yet another aspect the present invention provides a method for reducing or eliminating formation of a bacterial biofilm, wherein the bacteria have become resistant to treatment with at least one salicylamide compound or the combination of at least one salicylamide compound and at least one efflux pump inhibitor compound, wherein the salicylamide compound includes one or more nitro groups, comprising administering a nitro-prodrug antibiotic in an amount sufficient to reduce or eliminate formation of the biofilm.

In certain embodiments, the nitro-prodrug antibiotic is selected from the group consisting of nitrofurantoin, nitrofurazone, metronidazole, tinidazole, furazolidone, misonidazole, etanidazole, nifurtimox, ornidazole, benznidazole, dimetridazole, ronidazole, RSU-1069, RB-6145, CB1954, EF3, EF5, HX4 and fluorinated misonidazole.

### Definitions

The term “patient” includes human and non-human animals. Non-human animals include, but are not limited to, birds and mammals, in particular, mice, rabbits, cats, dogs, pigs, sheep, goats, cows, horses, and possums. The terms “patient” and “subject” are used interchangeably in this specification and in the context of preventing or treating a bacterial infection include medical health practitioners, as well as patients who are receiving treatment.

“Treatment” and like terms refer to methods and compositions to prevent, cure, or ameliorate a medical disease, disorder, or condition, and/or reduce at least a symptom of such disease or disorder. In particular, this includes methods and compositions to prevent or delay onset of a medical disease, disorder, or condition; to cure, correct, reduce, slow, or ameliorate the physical or developmental effects of a medical disease, disorder, or condition; and/or to prevent, end, reduce, or ameliorate the pain or suffering caused by the medical disease, disorder, or condition.

The term “preventing” means preventing in whole or in part, or ameliorating or controlling, or reducing or halting the production or occurrence of the thing or event, for example, the bacterial infection to be prevented.

The term “aryl” means an aromatic radical having 4 to 18 carbon atoms Examples include monocyclic groups, as well as fused groups such as bicyclic groups and tricyclic groups. Examples include phenyl, indenyl, 1-naphthyl, 2-naphthyl, azulenyl, heptalenyl, biphenyl, indacenyl, acenaphthyl, fluorenyl, phenalenyl, phenanthrenyl, anthracenyl, cyclopentacyclooctenyl, and benzocyclooctenyl.

The term “heteroaryl” means an aromatic radical having 4 to 18 carbon atoms and including one or more heteroatoms. Examples include monocyclic groups, as well as fused groups such as bicyclic groups and tricyclic groups. Examples include pyridyl, pyrrolyl, pyridazinyl, pyrimidinyl, pyrazinyl, quinazolinyl, quinolyl, isoquinolyl, quinoxalinyl, triazinyl, furyl, benzofuryl, isobenzofuryl, indolyl, thiophenyl, benzylthiophenyl, imidazolyl, benzimidazolyl, purinyl, pyrazolyl, indazolyl, oxazolyl, benzoxazolyl, isoxazole, benzisoxazolyl, triazolyl, thiazolyl, benzothiazolyl, and tetrazolyl.

The term “pharmaceutically acceptable salt” is intended to apply to non-toxic salts derived from inorganic or organic acids, including, for example, the following acid salts: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, p-toluenesulfonate, salicylate, succinate, sulfate, tartrate, thiocyanate and undecanoate.

As used in this specification, the words “comprises”, “comprising”, and similar words, are not to be interpreted in an exclusive or exhaustive sense. In other words, they are intended to mean “including, but not limited to.

For the purposes of the invention, any reference to the disclosed compounds includes all possible formulations, configurations, and conformations, for example, in free form (e.g. as a free acid or base), in the form of salts or hydrates, in the form of isomers (e.g. cis/trans isomers), stereoisomers such as enantiomers, diastereomers and epimers, in the form of mixtures of enantiomers or diastereomers, in the form of racemates or racemic mixtures, or in the form of individual enantiomers or diastereomers. Specific forms of the compounds are described in detail herein.

It will be appreciated that any of the sub-scopes disclosed herein, e.g. with respect to R¹, R², R³, R⁴, R⁵, R⁶, R⁷, R⁸, R⁹, R¹⁰, may be combined with any of the other sub-scopes disclosed herein to produce further sub-scopes.

It will also be appreciated that any reference to a range of numbers disclosed herein (e.g. 1 to 100) is intended to encompass all rational numbers within that range (e.g. 1.1, 20.5, 55.6, 70, 90) and also any range of rational numbers within that range (e.g. 1.1 to 3.5) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly disclosed herein.

### Examples

The invention is further described with reference to the following examples. It will be appreciated that the invention as claimed is not intended to be limited in any way by these examples.

Generation of E. coli Strains

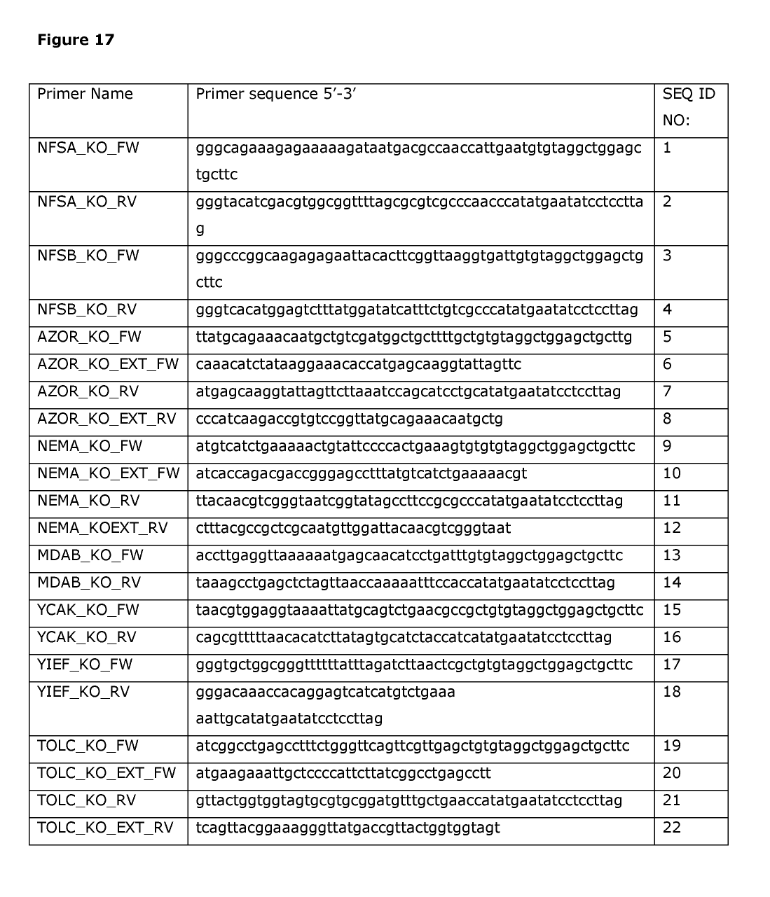

Deletion strains are generated by in-frame deletion using a PCR-amplified disruption cassette through the Red recombinase method (Datsenko, K. A. and Wanner, B. L. (2000). One-step inactivation of chromosomal genes in Escherichia coli K-12 using PCR products. Proc Natl Acad Sci USA 97:6640-6645.). PCR primers used for disruption cassette amplification and overlap PCR are depicted in FIG. 17. E. coli strain 7KO is derived from E. coli W3110 by deletion of the native nfsA, nfsB, azoR, nemA, yieF, ycaK and mdaB genes. E. coli strain 7KOΔtolC is derived from 7KO by deletion of the native tolC gene. E. coli strain 7KOΔtolC(DE3), which has an integrated ADE3 prophage to allow for inducible expression via a T7 RNA polymerase, is derived from 7KOΔtolC using a ADE3 lysogenization kit (Novagen, Merck, Darmstadt, Germany). Oxidoreductases are expressed in 7KOΔtolC from plasmid pUCX, an expression plasmid derived from pUC19 (Prosser, G. A., Copp, J. N., Mowday, A. M., Guise, C. P., Syddall, S. P., Williams, E. M., Horvat, C. N., Swe, P. S., Ashoorzadeh, A., Denny, W. A., Smaill, J. B., Patterson, A. V. and Ackerley, D. F. (2013). Creation and screening of a multi-family bacterial oxidoreductase library to discover novel nitroreductases that efficiently activate the bioreductive prodrugs CB1954 and PR-104A. Biochemical Pharmacology 85:1091-1103.). Oxidoreductases are expressed in 7KOΔtolC(DE3) from plasmid pET28 (Novagen, Merck, Darmstadt, Germany).

### Cell Sensitivity Assays

This procedure applies to all experiments yielding growth curves for Gram negative bacterial strains generated in the presence of niclosamide or 2-chloro-4-nitroaniline, unless noted otherwise below or in the individual Figure Descriptions. Individual wells of a 96-well microtitre plate containing 100 μL LB (supplemented with 100 μg mL⁻¹ ampicillin and 0.4% (w/v) glucose for 7KOΔtolC oxidoreductase-overexpressing strains) are inoculated in duplicate and incubated overnight at 30° C. with shaking at 200 rpm. The following day 100 μL of the overnight culture is used to inoculate 2 mL LB (supplemented with 50 μM IPTG for oxidoreductase-overexpressing strains, in addition to ampicillin and glucose as above) and incubated at 30° C., 200 rpm for 3.5 h. 40 μL aliquots from each culture are then added to individual wells of a sterile 384 well plate in duplicate, each containing a dilution series of niclosamide or 2-chloro-4-nitroaniline, including 0 μM controls in 40 μL of LB (supplemented with IPTG, ampicillin and glucose and antibiotics for oxidoreductase-overexpressing strains as above). Culture turbidity is monitored by optical density at 600 nm (OD600) 4 h post-challenge. Percentage growth is calculated by comparison of the niclosamide- or 2-chloro-4-nitroaniline-challenged cells with the unchallenged 0 μM controls, after subtracting the initial absorbance values (t=0 h). All microtitre plate absorbance readings are measured using an EnSpire™ 2300 Multilabel Reader (Perkin Elmer, Waltham, Mass.). Where given, ICso values (the drug concentrations that cause 50% growth inhibition for a given strain relative to the unchallenged control) are calculated using non-linear regression analysis in GraphPad Prism 5 (GraphPad Software, Inc., La Jolla, Calif.).

### 7KOΔtolC Oxidoreductase Library Niclosamide Growth Inhibition Assays

This procedure applies to the experiment depicted in FIG. 5. Individual wells of a 96-well microtitre plate containing 100 μL LB supplemented with 100 μg mL⁻¹ ampicillin and 0.4% (w/v) glucose are inoculated in duplicate with 7KOΔtolC library strains (including a pUCX empty plasmid control) and incubated overnight at 30° C. with shaking at 200 rpm. 100 μL of each overnight culture is used to inoculate 2 mL LB supplemented with 100 μg mL⁻¹ ampicillin and 50 μM IPTG and incubated at 30° C., 200 rpm for 3.5 h. 40 μL aliquots from each culture are then added to individual wells of a sterile 384 well plate, containing 40 μL LB medium supplemented with 100 μg mL⁻¹ ampicillin, 50 μM IPTG and either 2.5 μM niclosamide or control wells containing 0 μM niclosamide. Each oxidoreductase over-expression strain is tested in duplicate (independent replicates). Culture turbidity is monitored by optical density at 600 nm 4 h post-challenge. Percentage growth inhibition is calculated by comparison of the challenged cells with the unchallenged 0 μM controls, after subtracting the initial absorbance values (t=0 h), using the following formula: (1−(challenged OD600/unchallenged OD600))*100%. All microtitre plate absorbance readings are measured using an EnSpire™ 2300 Multilabel Reader (Perkin Elmer, Waltham, Mass.).

Analysis of the Ability of Niclosamide to Enrich for Clones Expressing Active Nitroreductase from a Variant Gene Library

This procedure applies to the experiments depicted in FIGS. 6 and 7. Electrocompetent SOS-R4 cells are transformed with a portion of the ˜95 million membered nfsA variant library that had been ligated into pUCX. Following transformation, cells are plated on agar plates containing 100 μg/mL ampicillin and 50 μg/mL spectinomycin, either with or without +0.5 μM niclosamide and 0.1 μM IPTG. Plates are grown overnight at 30° C. to allow colonies to form. 57 colonies are then picked from a plate of each media condition and (together with nfsA, empty plasmid, and media controls) transferred into 100 μl LB containing 100 μg/mL ampicillin and 50 μg/mL spectinomycin in the inner 60 wells of separate 96 well plates. Cultures are allowed to grow overnight before a glycerol stock is made from which to inoculate all subsequent assays. For the growth inhibition assays, each glycerol plate is used to inoculate the inner 60 wells of a fresh microtitre plate, each containing 150 μL of LB+100 μg/mL ampicillin, 50 μg/mL spectinomycin, and 0.4% glucose. The plates are then incubated overnight at 30° C., 200 rpm. The next day 15 μL of overnight culture is used to inoculate 200 μL of LB containing 100 μg/mL ampicillin, 50 μg/mL spectinomycin, 0.2% glucose, and 50 μM IPTG in each of the inner 60 wells of a fresh microtitre plate. Each plate is incubated for 2.5 h at 30° C., 200 rpm. 30 μL of each of these day cultures is then transferred to individual wells of a 384 well plate containing 30 μL of LB amended with 100 μg/mL ampicillin, 50 μg/mL spectinomycin, 0.2% glucose, and 50 μM IPTG as well as either 100 μM of prodrug (metronidazole for FIG. 6, tinidazole for FIG. 7) or equivalent DMSO vehicle-only control. Each selected clone is challenged in duplicate on each 384 well plate. The 384 well plate is then incubated for 3 h at 30° C., 200 rpm, after which the optical density at 600 nm of each well is read in the plate reader. Percentage growth inhibition is calculated by comparison of the challenged cells with the unchallenged 0 μM controls, after subtracting the initial absorbance values (t=0 h), using the following formula: (1−(challenged OD600/unchallenged OD600))*100%. All microtitre plate absorbance readings are measured using an EnSpire™ 2300 Multilabel Reader (Perkin Elmer, Waltham, Mass.).

### Heatmap Analysis of the Effect of Combined or Individual Niclosamide and PAβN Treatments on Different Bacterial Strains

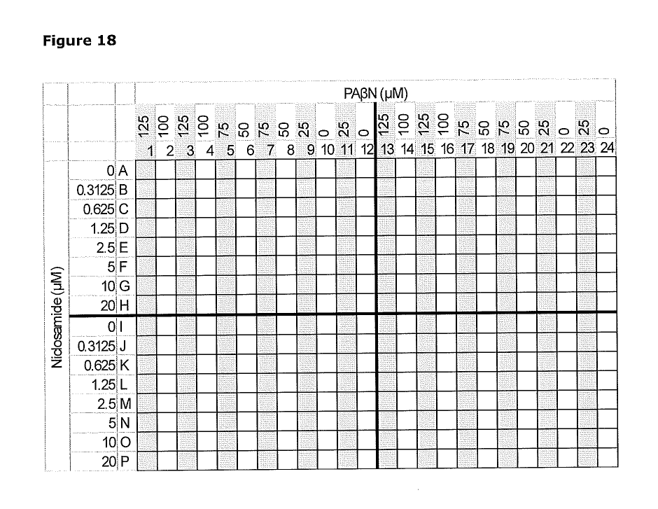

This procedure applies to the experiments depicted in FIGS. 8-15. The desired strain is inoculated into 3 mL LB and incubated for 16 hours at 30° C. (Psa-V or E. coli lab strains 7KO or 7KOΔtolC) or 37° C. (E. coli lab strain W3110, P. aeruginosa lab strain PAO1, or all clinical isolate strains), with shaking at 200 rpm. The OD600 of the overnight cultures is measured and the cells diluted in LB amended with 0.1 M MgSO₄ to an OD600 of 0.2, which would give a starting OD600 of 0.1 following a 1 in 2 dilution with media. Culture media (30 μL per well of a 384 well plate) contains LB, 0.1 M MgSO₄ and double the final desired concentration of PAβN to allow for a 1 in 2 dilution with bacterial culture. An aliquot of culture media (60 μL per well) for each PAβN dilution is supplemented with niclosamide at double the highest final concentration to be used to allow for the 1 in 2 dilution with bacterial culture. To row H (or P) of a 384 well plate, 60 μL of each niclosamide/PAβN media combination is added as shown in FIG. 18. To rows A-G, 30 μL of the culture media (PAβN only) is added. Serial dilution of the niclosamide is performed by removing 30 μL of media from row H and transferring it to row G, mixing, then transferring 30 μL of row G to row F and so on through to row B. After row B the final 30 μL is discarded and row A is left as 0 μM niclosamide. Each well is then inoculated with 30 μL of the bacterial culture OD600=0.2, to give a final OD600 of 0.1 per well. The OD600 is measured (t=0) and the cultures are incubated at 30° C. or 37° C. (as per overnight cultures), 200 rpm for 4 hours. The final OD600 (t=4) is then recorded. To calculate growth inhibition the t=0 value is subtracted from the t=4 value. The percentage growth is then calculated relative to the 0 μM niclosamide and 0 well μM PaβN well, which represents 100% growth.

### 7KOΔtolC(DE3) Cell Sensitivity Assays

This procedure applies to the experiment depicted in FIG. 16. Individual wells of a 96-well microtitre plate containing 100 μL LB supplemented with 50 μg mL⁻¹ are inoculated in duplicate with 7KOΔtolC(DE3) strains overexpressing E. coli nfsA, E. coli nfsB or a pET28 empty plasmid control. Strains are incubated overnight at 30° C. in a Heidolph titramax platform shaker at 900 rpm. 30 μL of each overnight culture is used to inoculate duplicate cultures of 600 μL LB supplemented with 50 μg mL⁻¹ kanamycin and 50 μM IPTG in a 96 well deep culture plate (Axygen). Cultures are incubated at 30° C., 1200 rpm for 2.5 h. 40 μL aliquots from each culture are then added to individual wells of a sterile 384 well plate, containing 40 μL LB medium supplemented with 100 μg mL⁻¹ kanamycin, 50 μM IPTG and serial dilutions of nitazoxanide, including 0 μM controls. Each nitroreductase over-expression strain is tested in duplicate (independent replicates). Culture turbidity is monitored by optical density at 600 nm 4 h post-challenge. Percentage growth values are calculated by comparison of the nitazoxanide-challenged cells with the unchallenged control, after subtracting the initial absorbance values (t=0 h). Microtitre plate absorbance readings are measured using an Eon Biotek Reader (BioTek Instruments Inc.).

### Growth Inhibition of Gram Positive Bacteria by Niclosamide or Nitazoxanide: IC₅₀ Measurements

This procedure applies to the experiments depicted in FIGS. 20 and 21. 15 mL tubes containing 2 mL tryptic soy broth (TSB) were inoculated with the Gram positive bacterial strains Staphylococcus aureus ATCC 43300 (MRSA), Bacillus thuringiensis P.1.IPS-80 serovar israelensis, or Listeria welshimeri ATCC 35897. Strains were incubated overnight at 37° C. with shaking at 200 rpm. The following day, the overnight cultures were diluted in fresh TSB to give an OD₆₀₀ of approximately 0.3. Duplicate 40 μL aliquots of this were then added to individual wells of a sterile 384 well plate, containing 40 μL TSB medium supplemented with serial dilutions of 2× niclosamide or nitazoxanide, including 0 μM controls. Culture turbidity was monitored by optical density at 600 nm at 4 h post-challenge. Percentage growth inhibition values were calculated by comparison of the niclosamide- or nitazoxanide-challenged cells with the unchallenged control for each strain, after subtracting the initial absorbance values (t=0 h). Microtitre plate absorbance readings were measured using an Enspire™ 2300 Multilabel Reader (Perkin Elmer, Waltham, Mass.). Inhibitory concentrations (IC₅₀; the concentration of compound at which growth of the test strain attains a level of turbidity 50% that of the unchallenged control) were calculated for each compound against each strain using Graphpad Prism 5 (GraphPad Software, Inc., La Jolla, Calif.).

The IC₅₀ values derived from the data presented in FIGS. 20 and 21 for the Gram positive strains S. aureus ATCC 43300, L. welshimeri ATCC 35897, and B. thuringiensis P.1.IPS-80 serovar israelensis across a range of concentrations of niclosamide or nitazoxanide, are presented in Table 1 as follows:

TABLE 2
IC₅₀ values for niclosamide and nitazoxanide against
Gram positive bacteria

| | Niclosamide | Nitazoxanide |
|---|---|---|
| Strain | (nM) | (nM) |
| Staphylococcus aureus ATCC 43300 | 132 | 2902 |
| (MRSA) | | |
| Listeria welshimeri ATCC 35897 | 284 | 8785 |
| Bacillus thuringiensis P.1.IPS-80 | 139 | 8671 |
| serovar israelensis | | |

Although the invention has been described by way of example, it should be appreciated that variations and modifications may be made without departing from the scope of the invention as defined in the claims. Furthermore, where known equivalents exist to specific features, such equivalents are incorporated as if specifically referred to in this specification.

1. A method for treating or preventing a bacterial infection in a patient, the method comprising administering to the patient, a salicylamide compound and an efflux pump inhibitor in amounts sufficient to treat or prevent the bacterial infection in the patient, wherein the bacterial infection is caused by Gram negative bacteria.

2. A method for reducing or eliminating formation of a bacterial biofilm comprising Gram negative bacteria, comprising administering a salicylamide compound and an efflux pump inhibitor in amounts sufficient to reduce or eliminate formation of the biofilm.

3. The method according to claims 1, 2 or 16, wherein the salicylamide compound is niclosamide or nitazoxanide.

4. The method according to any one of claims 1, 2 or 16, wherein the bacteria is selected from the group consisting of Klebsiella, Escherichia, Pseudomonas, Shigella, Salmonella, Acinetobacter, Neisseria and Burkholderia.

5. (canceled)

6. (canceled)

7. (canceled)

8. (canceled)

9. (canceled)

10. (canceled)

11. A method for protecting a bacterial cell against toxicity by at least one salicylamide compound, wherein the salicylamide compound includes one or more nitro groups, the method comprising increasing the expression and/or activity of at least one nitroreductase enzyme in the cell in an amount sufficient to protect against toxicity by the salicylamide compound.

12. A method for treating or preventing a bacterial infection in a patient or for preventing, reducing or eliminating formation of a bacterial biofilm, wherein the bacteria have become resistant to treatment with a nitro-prodrug antibiotic, comprising administering at least one salicylamide compound, wherein the salicylamide compound includes one or more nitro groups, in an amount sufficient to treat or prevent infection or to prevent, reduce or eliminate formation of the biofilm.

13. A method for treating or preventing a bacterial infection in a patient or for preventing, reducing or eliminating formation of a bacterial biofilm, wherein the bacteria have become resistant to treatment with at least one salicylamide compound or the combination of at least one salicylamide compound and at least one efflux pump inhibitor compound, wherein the salicylamide compound includes one or more nitro groups, comprising administering a nitro-prodrug antibiotic in an amount sufficient to treat or prevent the infection or to prevent, reduce or eliminate formation of the biofilm.

14. A composition comprising antibiotically effective amounts of:
(i) a salicylamide compound or a pharmaceutically acceptable salt thereof; and
(ii) an efflux pump inhibitor compound or a pharmaceutically acceptable salt thereof;
wherein the salicylamide compound and the efflux pump inhibitor are employed in proportions sufficient to produce a synergistic antibiotic effect.

15. A composition as claimed in claim 14, wherein:
(i) the salicylamide compound is niclosamide or nitazoxanide; and
(ii) the efflux pump inhibitor is a TolC efflux pump inhibitor.

16. A method for:
(i) treating or preventing a bacterial infection in a patient; or
(ii) reducing or eliminating formation of a bacterial biofilm
wherein the bacteria causing infection or biofilm formation comprises a TolC efflux pump, the method comprising the steps of administering to the patient or biofilm a salicylamide compound and a TolC efflux pump inhibitor in amounts sufficient to treat or prevent the bacterial infection in the patient or reduce or eliminate formation of the biofilm.

17. The method according to claim 1 or claim 2, wherein the efflux pump inhibitor is a TolC efflux pump inhibitor.

18. The method according to claim 17, wherein the TolC efflux pump inhibitor is phenylalanine-arginine β-napthylamide (PAβN), 2-3 dibromomaleimide or analogues thereof.

19. (canceled)


SEQUENCE LISTING (22 sequences)

SEQ ID NO: 1 - DNA, 60 bp, Artificial Sequence
  Synthetic Primer
gggcagaaagagaaaaagataatgacgccaaccattgaatgtgtaggctggagctgcttc

SEQ ID NO: 2 - DNA, 59 bp, Artificial Sequence
  Synthetic Primer
gggtacatcgacgtggcggttttagcgcgtcgcccaacccatatgaatatcctccttag

SEQ ID NO: 3 - DNA, 60 bp, Artificial Sequence
  Synthetic Primer
gggcccggcaagagagaattacacttcggttaaggtgattgtgtaggctggagctgcttc

SEQ ID NO: 4 - DNA, 59 bp, Artificial Sequence
  Synthetic Primer
gggtcacatggagtctttatggatatcatttctgtcgcccatatgaatatcctccttag

SEQ ID NO: 5 - DNA, 57 bp, Artificial Sequence
  Synthetic Primer
ttatgcagaaacaatgctgtcgatggctgcttttgctgtgtaggctggagctgcttg

SEQ ID NO: 6 - DNA, 41 bp, Artificial Sequence
  Synthetic Primer
caaacatctataaggaaacaccatgagcaaggtattagttc

SEQ ID NO: 7 - DNA, 56 bp, Artificial Sequence
  Synthetic Primer
atgagcaaggtattagttcttaaatccagcatcctgcatatgaatatcctccttag

SEQ ID NO: 8 - DNA, 39 bp, Artificial Sequence
  Synthetic Primer
cccatcaagaccgtgtccggttatgcagaaacaatgctg

SEQ ID NO: 9 - DNA, 57 bp, Artificial Sequence
  Synthetic Primer
atgtcatctgaaaaactgtattccccactgaaagtgtgtgtaggctggagctgcttc

SEQ ID NO: 10 - DNA, 42 bp, Artificial Sequence
  Synthetic Primer
atcaccagacgaccgggagcctttatgtcatctgaaaaacgt

SEQ ID NO: 11 - DNA, 56 bp, Artificial Sequence
  Synthetic Primer
ttacaacgtcgggtaatcggtatagccttccgcgcccatatgaatatcctccttag

SEQ ID NO: 12 - DNA, 41 bp, Artificial Sequence
  Synthetic Primer
ctttacgccgctcgcaatgttggattacaacgtcgggtaat

SEQ ID NO: 13 - DNA, 55 bp, Artificial Sequence
  Synthetic Primer
accttgaggttaaaaaatgagcaacatcctgatttgtgtaggctggagctgcttc

SEQ ID NO: 14 - DNA, 55 bp, Artificial Sequence
  Synthetic Primer
taaagcctgagctctagttaaccaaaaatttccaccatatgaatatcctccttag

SEQ ID NO: 15 - DNA, 57 bp, Artificial Sequence
  Synthetic Primer
taacgtggaggtaaaattatgcagtctgaacgccgctgtgtaggctggagctgcttc

SEQ ID NO: 16 - DNA, 56 bp, Artificial Sequence
  Synthetic Primer
cagcgtttttaacacatcttatagtgcatctaccatcatatgaatatcctccttag

SEQ ID NO: 17 - DNA, 58 bp, Artificial Sequence
  Synthetic Primer
gggtgctggcgggttttttatttagatcttaactcgctgtgtaggctggagctgcttc

SEQ ID NO: 18 - DNA, 57 bp, Artificial Sequence
  Synthetic Primer
gggacaaaccacaggagtcatcatgtctgaaaaattgcatatgaatatcctccttag

SEQ ID NO: 19 - DNA, 57 bp, Artificial Sequence
  Synthetic Primer
atcggcctgagcctttctgggttcagttcgttgagctgtgtaggctggagctgcttc

SEQ ID NO: 20 - DNA, 39 bp, Artificial Sequence
  Synthetic Primer
atgaagaaattgctccccattcttatcggcctgagcctt

SEQ ID NO: 21 - DNA, 56 bp, Artificial Sequence
  Synthetic Primer
gttactggtggtagtgcgtgcggatgtttgctgaaccatatgaatatcctccttag

SEQ ID NO: 22 - DNA, 39 bp, Artificial Sequence
  Synthetic Primer
tcagttacggaaagggttatgaccgttactggtggtagt